(19) United States
(12) Patent Application Publication
(10) Pub. No.: US 2005/0014291 A1
(43) Pub. Date: Jan. 20, 2005

(54) Assay method using biochemical analysis units and cleaning apparatus for the same

(75) Inventor: Nobuhiko Ogura, Kanagawa-ken (JP)

Correspondence Address: SUGHRUE MION, PLLC, 2100 PENNSYLVANIA AVENUE, N.W., SUITE 800, WASHINGTON, DC, 20037, US

(73) Assignee: FUJI PHOTO FILM CO., LTD.

(21) Appl. No.: 10/890,156

(22) Filed: Jul. 14, 2004

(30) Foreign Application Priority Data
Jul. 15, 2003 (JP) 196966/2003

(51) Int. Cl. G01N 33/543

(52) U.S. Cl. 436/518

(57) ABSTRACT

At a stage between a step for subjecting a receptor or a ligand to specific binding with ligands or receptors, each of which has been bound to one of porous adsorptive regions of a biochemical analysis unit, and a step for detecting the receptor or the ligand, which has been specifically bound to at least one of the ligands or at least one of the receptors, by the utilization of the labeling substance, at least one electrode is located such that the electrode is capable of causing an electric current to flow across each of the porous adsorptive regions of the biochemical analysis unit. A voltage is applied to the electrode in order to cause the electric current to flow across each of the porous adsorptive regions of the biochemical analysis unit.

### BACKGROUND OF THE INVENTION

1. Field of the Invention

This invention relates to an assay method using a biochemical analysis unit provided with a plurality of porous adsorptive regions, to which ligands or receptors have been bound respectively. This invention also relates to a cleaning apparatus for a biochemical analysis unit.

2. Description of the Related Art

With micro array analysis systems and macro array analysis systems, various assay techniques using biochemical analysis units have heretofore been proposed. With the assay techniques using the biochemical analysis units, liquids containing ligands or receptors (i.e., the substances, which are capable of specifically binding to organism-originating substances and whose base sequences, base lengths, compositions, characteristics, and the like, are known) are spotted onto different positions on a surface of a biochemical analysis unit, such as a membrane filter, i.e. onto adsorptive regions of the biochemical analysis unit, and the ligands or the receptors are thus bound respectively to the adsorptive regions of the biochemical analysis unit. Examples of the ligands or the receptors include hormones, tumor markers, enzymes, antibodies, antigens, abzymes, other proteins, nucleic acids, cDNA's, DNA's, and RNA'S. Thereafter, a labeled receptor or a labeled ligand, which has been labeled with a labeling substance, is subjected to specific binding with the ligands or the receptors, which have been bound to the adsorptive regions of the biochemical analysis unit. The labeled receptor or the labeled ligand is thus specifically bound to at least one of the ligands or the receptors, which have been bound to the adsorptive regions of the biochemical analysis unit. The labeled receptor or the labeled ligand is the substance, which has been sampled from an organism through extraction, isolation, or the like, or has been subjected to chemical treatment after being sampled, and which has been labeled with the radioactive labeling substance, the fluorescent labeling substance, the labeling substance capable of causing a chemical luminescence substrate to produce the chemical luminescence when being brought into contact with the chemical luminescence substrate, or the like. Examples of the labeled receptors or the labeled ligands include hormones, tumor markers, enzymes, antibodies, antigens, abzymes, other proteins, nucleic acids, DNA's, andmRNA's. Examples of the labeling substances include a radioactive labeling substance, a fluorescent labeling substance, and a chemical luminescent labeling substance capable of producing chemical luminescence when being brought into contact with a chemical luminescence substrate.

In cases where the labeled receptor or the labeled ligand has been labeled with the radioactive labeling substance, a stimulable phosphor layer of a stimulable phosphor sheet is then exposed to radiation radiated out from the radioactive labeling substance, which is contained selectively in the adsorptive regions of the biochemical analysis unit. Thereafter, the stimulable phosphor layer is exposed to stimulating rays, which cause the stimulable phosphor layer to emit light in proportion to the amount of energy stored on the stimulable phosphor layer during the exposure of the stimulable phosphor layer to the radiation. The light emitted by the stimulable phosphor layer is detected photoelectrically. In this manner, the labeled receptor or the labeled ligand having been specifically bound to at least one of the ligands or the receptors, which have been bound to the adsorptive regions of the biochemical analysis unit, is detected.

In cases where the labeled receptor or the labeled ligand has been labeled with the fluorescent labeling substance, excitation light is irradiated to the adsorptive regions of the biochemical analysis unit, and the fluorescent labeling substance, which is contained selectively in the adsorptive regions of the biochemical analysis unit, is excited by the excitation light to produce fluorescence. The thus produced fluorescence is detected photoelectrically.

In cases where the labeled receptor or the labeled ligand has been labeled with the chemical luminescent labeling substance capable of producing the chemical luminescence when being brought into contact with a chemical luminescence substrate, the chemical luminescent labeling substance, which is contained selectively in the adsorptive regions of the biochemical analysis unit, is brought into contact with the chemical luminescence substrate. Also, the chemical luminescence produced by the chemical luminescent labeling substance is detected photoelectrically. (The assay techniques using the biochemical analysis units are described in, for example, U.S. Patent Laid-Open No. 20020016009.)

With the assay techniques described above, a large number of the adsorptive regions, to which the ligands or the receptors are bound, are capable of being formed at a high density at different positions on the surface of the biochemical analysis unit, and the labeled receptor or the labeled ligand, which has been labeled with the labeling substance, is capable of being subjected to the specific binding with the ligands or the receptors, which have been bound to the adsorptive regions formed at a high density at different positions on the surface of the biochemical analysis unit. Therefore, the assay techniques described above have the advantages in that a receptor or a ligand is capable of being analyzed quickly.

As one of the assay techniques using the biochemical analysis units, for example, a gene expression analysis technique using a DNA micro array has heretofore been known. With the gene expression analysis technique using the DNA micro array, firstly, DNA probes, which are different kinds of known genes, are fixed respectively to the plurality of the adsorptive regions of the biochemical analysis unit, which adsorptive regions are constituted of membranes, or the like. A hybridization liquid containing a target DNA to be detected (e.g., a DNA having been labeled with a fluorescent substance) is subjected to a reaction with the DNA probes, which have been fixed respectively to the adsorptive regions of the biochemical analysis unit. In cases where the target DNA has a base sequence, which is complementary to the base sequence of one of the probe DNA's, the target DNA undergoes the specific binding with the probe DNA. Surplus target DNA, which has not been bound to the probe DNA, is then washed off. Also, the adsorptive regions are scanned by use of a detecting apparatus utilizing a laser. In cases where the target DNA has been labeled with the fluorescent substance, the fluorescence is produced from the adsorptive region containing the probe DNA to which the target DNA has been bound. The intensity of the thus produced fluorescence is detected. The bound gene expression level is capable of being analyzed in accordance with the intensity of the fluorescence. The specific binding of the DNA's, which have the base sequences complementary to each other, with each other is ordinarily referred to as the hybridization. Conditions (such as the temperature and the salt concentration) optimum for the hybridization may vary for the different kinds of the probe DNA's having been fixed respectively to the adsorptive regions. However, it is not always possible to perform the reaction under the conditions optimum for each of the probe DNA's, which have been fixed respectively to the adsorptive regions having been located at a high density. Therefore, ordinarily, the reaction is performed under the identical conditions with respect to all of the adsorptive regions. Accordingly, it may often occur that a target DNA, which does not have the characteristics of undergoing the hybridization with the probe DNA's having been fixed to the adsorptive regions, undergoes non-specific binding with the probe DNA's having been fixed to the adsorptive regions. The non-specific binding described above is referred to as the cross hybridization.

With the conventional assay techniques, after the hybridization liquid containing the target DNA has been subjected to the reaction with the biochemical analysis unit, to which the probe DNA's have been fixed, a liquid washing operation utilizing a washing liquid is performed in order to remove the surplus target DNA, which has not been bound to the probe DNA and remains in the adsorptive regions. However, the target DNA, which has undergone the cross hybridization with the probe DNA's having been fixed to the adsorptive regions, has been bound to the probe DNA's partially or by a certain kind of interaction. Therefore, with the conventional washing technique utilizing the liquid washing, it is not always possible to achieve uniform control of the washing intensity, and accurate washing is not capable of being performed. As a result, in the assay techniques described above, the target DNA, which has undergone the cross hybridization with the probe DNA's having been fixed to the adsorptive regions, causes noise to occur at the time of the detection, and the detection accuracy is not capable of being kept high.

As described above, the receptor or the ligand (in the example described above, the target DNA having undergone the cross hybridization), which has undergone the non-specific binding with the ligands or the receptors having been bound to the adsorptive regions, causes noise to occur at the time of the detection and causes the detection accuracy to become low. The state, in which the receptor or the ligand has undergone the non-specific binding with the ligands or the receptors having been bound to the adsorptive regions, is the state, in which the receptor or the ligand has not been bound at certain sites to the ligand or the receptor having been fixed to an adsorptive region, or the state, in which the receptor or the ligand has been bound to the ligand or the receptor merely by a certain kind of interaction other than the specific binding, instead of being bound perfectly. It has been known that, as in the cases of the cross hybridization occurring during the hybridization, the non-specific binding may occur during various kinds of specific binding, such as an antigen-antibody reaction, an antibody-antibody reaction, and the binding of an auxiliary substance and an auxiliary substance-combinable labeling substance with each other.

Commercially available micro arrays are designed such that the ligands or the receptors having been bound to the adsorptive regions may not be apt to undergo the non-specific binding. However, it is not always possible to design the micro arrays such that the ligands or the receptors having been bound to the adsorptive regions do not in the least undergo the non-specific binding. In particular, with respect to a long gene, such as a cDNA, it is almost impossible to design the micro arrays such that the non-specific binding (i.e., the cross hybridization) does not occur. Also, in cases where research workers makes experiments by binding the ligands or the receptors with micro arrays, considerable time and labor are required to design the ligands or the receptors such that the non-specific binding may be eliminated.

As a technique for suppressing the cross hybridization, a technique for using a unit, in which a gene is fitted onto an electrode, is disclosed in U.S. Pat. No. 5,605,662 described above. The disclosed technique aims at suppressing the cross hybridization by the utilization the characteristics in that the gene is charged negatively. However, with the disclosed technique, the cost of the unit combined with the electrode into an integral body is high. Also, with the disclosed technique, commercially available micro arrays and micro arrays prepared by research workers are not capable of being utilized. Further, with commercially available array chips, in which oligo-substances are fixed onto electrodes, it is not always possible to set an optimum electric field for each of the electrodes due to adverse effects of an adjacent electrode.

### SUMMARY OF THE INVENTION

The primary object of the present invention is to provide an assay method using a biochemical analysis unit, wherein a non-specifically bound substance, such as a receptor or a ligand having been bound non-specifically, is capable of being removed.

Another object of the present invention is to provide a cleaning apparatus for a biochemical analysis unit, wherein a non-specifically bound substance, such as a receptor or a ligand having been bound non-specifically, is capable of being removed.

The present invention provides a first assay method using a biochemical analysis unit, comprising the steps of:
i) obtaining a biochemical analysis unit provided with a plurality of porous adsorptive regions, to which ligands or receptors have been bound respectively,
ii) subjecting at least one kind of a receptor or at least one kind of a ligand to specific binding with the ligands or the receptors, each of which has been bound to one of the porous adsorptive regions of the biochemical analysis unit, the receptor or the ligand being thereby specifically bound to at least one of the ligands, each of which has been bound to one of the porous adsorptive regions of the biochemical analysis unit, or at least one of the receptors, each of which has been bound to one of the porous adsorptive regions of the biochemical analysis unit, and
iii) detecting the receptor or the ligand, which has thus been specifically bound to at least one of the ligands or at least one of the receptors, by the utilization of a labeling substance,
wherein, at a stage between the step for subjecting the receptor or the ligand to the specific binding with the ligands or the receptors, each of which has been bound to one of the porous adsorptive regions of the biochemical analysis unit, and the step for detecting the receptor or the ligand, which has been specifically bound to at least one of the ligands or at least one of the receptors, by the utilization of the labeling substance,
at least one electrode is located such that the electrode is capable of causing an electric current to flow across each of the porous adsorptive regions of the biochemical analysis unit, and
a voltage is applied to the electrode in order to cause the electric current to flow across each of the porous adsorptive regions of the biochemical analysis unit.

The present invention also provides a second assay method using a biochemical analysis unit, comprising the steps of:
i) obtaining a biochemical analysis unit provided with a plurality of porous adsorptive regions, to which ligands or receptors have been bound respectively,
ii) subjecting at least one kind of an auxiliary substance-bound receptor or at least one kind of an auxiliary substance-bound ligand, to which an auxiliary substance has been bound, to specific binding with the ligands or the receptors, each of which has been bound to one of the porous adsorptive regions of the biochemical analysis unit, the auxiliary substance-bound receptor or the auxiliary substance-bound ligand being thereby specifically bound to at least one of the ligands, each of which has been bound to one of the porous adsorptive regions of the biochemical analysis unit, or at least one of the receptors, each of which has been bound to one of the porous adsorptive regions of the biochemical analysis unit,
iii) subjecting an auxiliary substance-combinable labeling substance, which is capable of undergoing specific binding with the auxiliary substance, to specific binding with the auxiliary substance of the auxiliary substance-bound receptor or the auxiliary substance-bound ligand having been specifically bound to at least one of the ligands, each of which has been bound to one of the porous adsorptive regions of the biochemical analysis unit, or at least one of the receptors, each of which has been bound to one of the porous adsorptive regions of the biochemical analysis unit, and
iv) detecting the auxiliary substance-bound receptor or the auxiliary substance-bound ligand, which has been specifically bound to at least one of the ligands or at least one of the receptors, by the utilization of the auxiliary substance-combinable labeling substance,
wherein, at a stage between the step for subjecting the auxiliary substance-bound receptor or the auxiliary substance-bound ligand to the specific binding with the ligands or the receptors, each of which has been bound to one of the porous adsorptive regions of the biochemical analysis unit, and the step for subjecting the auxiliary substance-combinable labeling substance to the specific binding with the auxiliary substance of the auxiliary substance-bound receptor or the auxiliary substance-bound ligand having been specifically bound to at least one of the ligands or at least one of the receptors, and/or
at a stage between the step for subjecting the auxiliary substance-combinable labeling substance to the specific binding with the auxiliary substance of the auxiliary substance-bound receptor or the auxiliary substance-bound ligand having been specifically bound to at least one of the ligands or at least one of the receptors, and the step for detecting the auxiliary substance-bound receptor or the auxiliary substance-bound ligand, which has been specifically bound to at least one of the ligands or at least one of the receptors, by the utilization of the auxiliary substance-combinable labeling substance,
at least one electrode is located such that the electrode is capable of causing an electric current to flow across each of the porous adsorptive regions of the biochemical analysis unit, and
a voltage is applied to the electrode in order to cause the electric current to flow across each of the porous adsorptive regions of the biochemical analysis unit.

In the second assay method using a biochemical analysis unit in accordance with the present invention, the operation for locating at least one electrode, such that the electrode is capable of causing the electric current to flow across each of the porous adsorptive regions of the biochemical analysis unit, and applying the voltage to the electrode in order to cause the electric current to flow across each of the porous adsorptive regions of the biochemical analysis unit may be performed at the stage between the step for subjecting the auxiliary substance-bound receptor or the auxiliary substance-bound ligand to the specific binding with the ligands or the receptors, each of which has been bound to one of the porous adsorptive regions of the biochemical analysis unit, and the step for subjecting the auxiliary substance-combinable labeling substance to the specific binding with the auxiliary substance of the auxiliary substance-bound receptor or the auxiliary substance-bound ligand having been specifically bound to at least one of the ligands or at least one of the receptors. Alternatively, the operation for locating at least one electrode, such that the electrode is capable of causing the electric current to flow across each of the porous adsorptive regions of the biochemical analysis unit, and applying the voltage to the electrode in order to cause the electric current to flow across each of the porous adsorptive regions of the biochemical analysis unit may be performed at the stage between the step for subjecting the auxiliary substance-combinable labeling substance to the specific binding with the auxiliary substance of the auxiliary substance-bound receptor or the auxiliary substance-bound ligand having been specifically bound to at least one of the ligands or at least one of the receptors, and the step for detecting the auxiliary substance-bound receptor or the auxiliary substance-bound ligand, which has been specifically bound to at least one of the ligands or at least one of the receptors, by the utilization of the auxiliary substance-combinable labeling substance. As another alternative, the operation for locating at least one electrode, such that the electrode is capable of causing the electric current to flow across each of the porous adsorptive regions of the biochemical analysis unit, and applying the voltage to the electrode in order to cause the electric current to flow across each of the porous adsorptive regions of the biochemical analysis unit may be performed at both the stages described above.

The present invention further provides a cleaning apparatus for cleaning a biochemical analysis unit provided with a plurality of porous adsorptive regions, to which ligands or receptors have been bound respectively, at least one kind of a receptor or at least one kind of a ligand having been specifically bound to at least one of the ligands, each of which has been bound to one of the porous adsorptive regions of the biochemical analysis unit, or at least one of the receptors, each of which has been bound to one of the porous adsorptive regions of the biochemical analysis unit, the apparatus comprising:
i) at least one electrode, which is located such that the electrode is capable of causing an electric current to flow across each of the porous adsorptive regions of the biochemical analysis unit, and
ii) control means for applying a voltage to the electrode in order to cause the electric current to flow across each of the porous adsorptive regions of the biochemical analysis unit.

The term “at least one kind of a receptor or at least one kind of a ligand having been specifically bound to at least one of ligands or at least one of receptors” as used herein for the cleaning apparatus for a biochemical analysis unit in accordance with the present invention embraces the auxiliary substance-bound receptor or the auxiliary substance-bound ligand, and the like.

The term “cleaning a biochemical analysis unit” as used herein for the cleaning apparatus for a biochemical analysis unit in accordance with the present invention means that the receptor or the ligand, which has not been bound to the ligands or the receptors having been bound to the adsorptive regions of the biochemical analysis unit, is removed by cleaning.

In the assay method using a biochemical analysis unit in accordance with the present invention and the cleaning apparatus for a biochemical analysis unit in accordance with the present invention, at least one electrode is located such that the electrode is capable of causing the electric current to flow across each of the porous adsorptive regions of the biochemical analysis unit. Also, the voltage is applied to the electrode in order to cause the electric current to flow across each of the porous adsorptive regions of the biochemical analysis unit. Specifically, a plurality of electrodes may be located, such that each of the electrodes is associated with one of the all adsorptive regions of the biochemical analysis unit or one of several adsorptive regions among the all adsorptive regions of the biochemical analysis unit. Also, the voltage may be applied to all of the electrodes simultaneously, and the electric current may thus be caused to flow simultaneously across each of the plurality of the adsorptive regions. Alternatively, a plurality of electrodes may be located, such that each of the electrodes is associated with one of the plurality of the adsorptive regions of the biochemical analysis unit, and the voltage may be applied to each of the electrodes successively. The electric current may thus be caused to flow successively across each of the plurality of the adsorptive regions. The electric current may thus be caused to flow ultimately across all of the plurality of the adsorptive regions. As another alternative, a plurality of electrodes may be located, such that each of the electrodes is associated with one of the plurality of the adsorptive regions of the biochemical analysis unit, and the voltage may be applied to the electrodes one by one. The electric current may thus be caused to flow across each of certain selected adsorptive regions among the plurality of the adsorptive regions. As a further alternative, only one electrode may be utilized and moved to each of the plurality of the adsorptive regions successively. The voltage may be applied to the electrode, and the electric current may thus be caused to flow successively across each of the plurality of the adsorptive regions. As a still further alternative, only one electrode may be utilized and moved to each of certain selected adsorptive regions among the plurality of the adsorptive regions successively. The voltage may be applied to the electrode, and the electric current may thus be caused to flow successively across each of the certain selected adsorptive regions among the plurality of the adsorptive regions.

Also, the value of the voltage applied to the electrode may be adjusted for each of the adsorptive regions. Further, the biochemical analysis unit may be electrically connected to the ground.

The receptor or the ligand subjected to the specific binding with the ligands or the receptors, each of which has been bound to one of the porous adsorptive regions of the biochemical analysis unit, is the substance, which has been sampled from an organism through extraction, isolation, or the like, or has been subjected to chemical treatment after being sampled. Examples of the receptors or the ligands include hormones, tumor markers, enzymes, antibodies, antigens, abzymes, other proteins, nucleic acids, DNA's, and mRNA's. Ordinarily, the substance acting as the receptor or the ligand has positive or negative electric charges. Therefore, in cases where an electric field is applied to the liquid, which contains the substance acting as the receptor or the ligand, in each of the adsorptive regions of the biochemical analysis unit, the substance moves toward the electrode. However, the receptor or the ligand, which has been specifically bound to at least one of the ligands or the receptors having been bound respectively to the adsorptive regions of the biochemical analysis unit, has been bound with a large binding force. Therefore, in cases where the electric field is applied to the liquid, which contains the receptor or the ligand, in each of the adsorptive regions of the biochemical analysis unit, the receptor or the ligand, which has been specifically bound to at least one of the ligands or the receptors having been bound respectively to the adsorptive regions of the biochemical analysis unit, does not readily separate from the ligand or the receptor having been bound to the corresponding adsorptive region of the biochemical analysis unit. However, in cases where the electric field is applied to the liquid, which contains the receptor or the ligand, in each of the adsorptive regions of the biochemical analysis unit, the surplus receptor or the surplus ligand, which has not been specifically bound to the ligands or the receptors having been bound respectively to the adsorptive regions of the biochemical analysis unit, moves toward the electrode. Also, the receptor or the ligand, which has been non-specifically bound to the ligands or the receptors having been bound respectively to the adsorptive regions of the biochemical analysis unit, has been bound with a small binding force. Therefore, in cases where the electric field is applied to the liquid, which contains the receptor or the ligand, in each of the adsorptive regions of the biochemical analysis unit, the receptor or the ligand, which has been non-specifically bound to the ligand or the receptor having been bound respectively to the adsorptive regions of the biochemical analysis unit, separates from the ligand or the receptor having been bound to the corresponding adsorptive region of the biochemical analysis unit and moves toward the electrode.

Examples of the auxiliary substances include antigens, such as digoxigenin, biotin, avidin, and fluorescein, and antibodies with respect to the above-enumerated antigens. The auxiliary substance-combinable labeling substance is the substance capable of undergoing the specific binding with the auxiliary substance. Ordinarily, the auxiliary substance-combinable labeling substance, or the like, has positive or negative electric charges. Therefore, in cases where the electric field is applied to the liquid, which contains the auxiliary substance-combinable labeling substance, in each of the adsorptive regions of the biochemical analysis unit, the auxiliary substance-combinable labeling substance, which has not been specifically bound to the auxiliary substance of the auxiliary substance-bound receptor or the auxiliary substance-bound ligand having been specifically bound to the ligand or the receptor, moves toward the electrode.

With the first assay method using a biochemical analysis unit in accordance with the present invention, after the biochemical analysis unit provided with the plurality of the porous adsorptive regions, to which the ligands or the receptors have been bound respectively, is obtained, at least one kind of the receptor or at least one kind of the ligand is subjected to the specific binding with the ligands or the receptors, each of which has been bound to one of the porous adsorptive regions of the biochemical analysis unit. The receptor or the ligand is thereby specifically bound to at least one of the ligands, each of which has been bound to one of the porous adsorptive regions of the biochemical analysis unit, or at least one of the receptors, each of which has been bound to one of the porous adsorptive regions of the biochemical analysis unit. Also, the receptor or the ligand, which has thus been specifically bound to at least one of the ligands or at least one of the receptors, is detected by the utilization of a labeling substance. At the stage between the step for subjecting the receptor or the ligand to the specific binding with the ligands or the receptors, each of which has been bound to one of the porous adsorptive regions of the biochemical analysis unit, and the step for detecting the receptor or the ligand, which has been specifically bound to at least one of the ligands or at least one of the receptors, by the utilization of the labeling substance, at least one electrode is located such that the electrode is capable of causing the electric current to flow across each of the porous adsorptive regions of the biochemical analysis unit, and the voltage is applied to the electrode in order to cause the electric current to flow across each of the porous adsorptive regions of the biochemical analysis unit. Therefore, with the first assay method using a biochemical analysis unit in accordance with the present invention, the receptor or the ligand, which has been non-specifically bound to the ligands or the receptors having been bound respectively to the adsorptive regions of the biochemical analysis unit, and which was not capable of being removed easily with conventional liquid washing techniques, is capable of being removed easily, and the detection accuracy is capable of being enhanced.

With the second assay method using a biochemical analysis unit in accordance with the present invention, after the biochemical analysis unit provided with the plurality of the porous adsorptive regions, to which the ligands or the receptors have been bound respectively, is obtained, at least one kind of the auxiliary substance-bound receptor or at least one kind of the auxiliary substance-bound ligand, to which the auxiliary substance has been bound, is subjected to the specific binding with the ligands or the receptors, each of which has been bound to one of the porous adsorptive regions of the biochemical analysis unit. The auxiliary substance-bound receptor or the auxiliary substance-bound ligand is thereby specifically bound to at least one of the ligands, each of which has been bound to one of the porous adsorptive regions of the biochemical analysis unit, or at least one of the receptors, each of which has been bound to one of the porous adsorptive regions of the biochemical analysis unit. Also, the auxiliary substance-combinable labeling substance, which is capable of undergoing the specific binding with the auxiliary substance, is subjected to the specific binding with the auxiliary substance of the auxiliary substance-bound receptor or the auxiliary substance-bound ligand having been specifically bound to at least one of the ligands, each of which has been bound to one of the porous adsorptive regions of the biochemical analysis unit, or at least one of the receptors, each of which has been bound to one of the porous adsorptive regions of the biochemical analysis unit. Further, the auxiliary substance-bound receptor or the auxiliary substance-bound ligand, which has been specifically bound to at least one of the ligands or at least one of the receptors, is detected by the utilization of the auxiliary substance-combinable labeling substance. At the stage between the step for subjecting the auxiliary substance-bound receptor or the auxiliary substance-bound ligand to the specific binding with the ligands or the receptors, each of which has been bound to one of the porous adsorptive regions of the biochemical analysis unit, and the step for subjecting the auxiliary substance-combinable labeling substance to the specific binding with the auxiliary substance of the auxiliary substance-bound receptor or the auxiliary substance-bound ligand having been specifically bound to at least one of the ligands or at least one of the receptors, at least one electrode is located such that the electrode is capable of causing the electric current to flow across each of the porous adsorptive regions of the biochemical analysis unit, and the voltage is applied to the electrode in order to cause the electric current to flow across each of the porous adsorptive regions of the biochemical analysis unit. Therefore, with the second assay method using a biochemical analysis unit in accordance with the present invention, the auxiliary substance-bound receptor or the auxiliary substance-bound ligand, which has been non-specifically bound to the ligands or the receptors having been bound respectively to the adsorptive regions of the biochemical analysis unit, and which was not capable of being removed easily with the conventional liquid washing techniques, is capable of being removed easily, and the detection accuracy is capable of being enhanced.

With the second assay method using a biochemical analysis unit in accordance with the present invention, at the stage between the step for subjecting the auxiliary substance-combinable labeling substance to the specific binding with the auxiliary substance of the auxiliary substance-bound receptor or the auxiliary substance-bound ligand having been specifically bound to at least one of the ligands or at least one of the receptors, and the step for detecting the auxiliary substance-bound receptor or the auxiliary substance-bound ligand, which has been specifically bound to at least one of the ligands or at least one of the receptors, by the utilization of the auxiliary substance-combinable labeling substance, at least one electrode may be located such that the electrode is capable of causing the electric current to flow across each of the porous adsorptive regions of the biochemical analysis unit, and the voltage may be applied to the electrode in order to cause the electric current to flow across each of the porous adsorptive regions of the biochemical analysis unit. In such cases, the auxiliary substance-combinable labeling substance, which has been non-specifically bound to the auxiliary substance of the auxiliary substance-bound receptor or the auxiliary substance-bound ligand having been specifically bound to the ligands or the receptors having been bound respectively to the adsorptive regions of the biochemical analysis unit, and which was not capable of being removed easily with the conventional liquid washing techniques, is capable of being removed easily, and the detection accuracy is capable of being enhanced.

It may often occur that the auxiliary substance-combinable labeling substance, or the like, clings to the adsorptive regions of the biochemical analysis unit and is not capable of being removed easily with the ordinary liquid washing. However, with the second assay method using a biochemical analysis unit in accordance with the present invention, wherein the electric current is caused to flow across each of the adsorptive regions of the biochemical analysis unit, the auxiliary substance-combinable labeling substance, or the like, which has clung to the adsorptive regions of the biochemical analysis unit, is capable of being removed easily.

The cleaning apparatus in accordance with the present invention is used in order to clean the biochemical analysis unit provided with the plurality of the porous adsorptive regions, to which the ligands or the receptors have been bound respectively, at least one kind of the receptor or at least one kind of the ligand having been specifically bound to at least one of the ligands, each of which has been bound to one of the porous adsorptive regions of the biochemical analysis unit, or at least one of the receptors, each of which has been bound to one of the porous adsorptive regions of the biochemical analysis unit. The cleaning apparatus for a biochemical analysis unit in accordance with the present invention comprises at least one electrode, which is located such that the electrode is capable of causing the electric current to flow across each of the porous adsorptive regions of the biochemical analysis unit, and the control means for applying the voltage to the electrode in order to cause the electric current to flow across each of the porous adsorptive regions of the biochemical analysis unit. Therefore, with the cleaning apparatus for a biochemical analysis unit in accordance with the present invention, the non-specifically bound substance, such as the receptor or the ligand having been bound non-specifically, which was not capable of being removed easily with conventional washing apparatuses for performing the liquid washing, is capable of being caused to move toward the electrode and is capable of being removed easily.

The first and second assay methods using a biochemical analysis unit in accordance with the present invention and the cleaning apparatus for a biochemical analysis unit in accordance with the present invention maybe modified such that the value of the voltage applied to the electrode is adjusted for each of the adsorptive regions. In such cases, the value of the voltage applied to the electrode or the value of the electric current flowing across each of the adsorptive regions of the biochemical analysis unit is capable of being adjusted in accordance with the characteristics of the non-specifically bound substance, such as the receptor or the ligand having been bound non-specifically to the ligand or the receptor having been bound to each of the adsorptive regions of the biochemical analysis unit. Therefore, the detection accuracy is capable of being enhanced even further.

Also, in cases where the electrode is located such that the electrode is capable of causing the electric current to flow across each of the porous adsorptive regions of the biochemical analysis unit, and the voltage is applied to the electrode in order to cause the electric current to flow across each of the porous adsorptive regions of the biochemical analysis unit, the biochemical analysis unit may be electrically connected to the ground. In such cases, adverse effects of the electric field formed in the adsorptive region associated with the electrode, to which the voltage is applied, upon the adsorptive region associated with the electrode, to which the voltage is not applied, are capable of being minimized.

### BRIEF DESCRIPTION OF THE DRAWINGS

FIG. 1 is a schematic perspective view showing an example of a biochemical analysis unit utilized for the assay method using a biochemical analysis unit in accordance with the present invention,

FIG. 2 is a schematic sectional view showing an example of a reaction vessel utilized for subjecting a receptor or a ligand to a reaction with ligands or receptors, each of which has been bound to one of porous adsorptive regions of the biochemical analysis unit,

FIG. 3 is a schematic sectional view showing a first embodiment of the cleaning apparatus for a biochemical analysis unit in accordance with the present invention,

FIG. 4 is a schematic bottom view showing an electric field forming device, which is employed in the first embodiment of the cleaning apparatus for a biochemical analysis unit in accordance with the present invention,

FIG. 5 is a schematic sectional view showing each of electrodes, which are employed in the first embodiment of the cleaning apparatus for a biochemical analysis unit in accordance with the present invention,

FIG. 6 is a block diagram showing a control system, an actuating system, and an input system of the electric field forming device, which is employed in the first embodiment of the cleaning apparatus for a biochemical analysis unit in accordance with the present invention,

FIG. 7 is an explanatory view showing how cross hybridization of DNA's occur,

FIG. 8A is an explanatory view showing how a receptor or a ligand is non-specifically bound to ligands or receptors, each of which has been bound to one of porous adsorptive regions of the biochemical analysis unit,

FIG. 8B is an explanatory view showing how the receptor or the ligand having been non-specifically bound to the ligands or the receptors, each of which has been bound to one of porous adsorptive regions of the biochemical analysis unit, separates from the ligands or the receptors,

FIG. 9 is a schematic sectional view showing a second embodiment of the cleaning apparatus for a biochemical analysis unit in accordance with the present invention,

FIG. 10 is a block diagram showing a control system, an actuating system, and an input system of an electric field forming device, which is employed in the second embodiment of the cleaning apparatus for a biochemical analysis unit in accordance with the present invention,

FIG. 11 is a schematic sectional view showing an electrode of an electric field forming device, which is employed in a third embodiment of the cleaning apparatus for a biochemical analysis unit in accordance with the present invention,

FIG. 12 is a schematic plan view showing the third embodiment of the cleaning apparatus for a biochemical analysis unit in accordance with the present invention,

FIG. 13 is a block diagram showing a control system, an actuating system, an input system, and a detection system of an electric field forming device, which is employed in the third embodiment of the cleaning apparatus for a biochemical analysis unit in accordance with the present invention,

FIG. 14 is a schematic sectional view showing a modification of the first embodiment of the cleaning apparatus for a biochemical analysis unit in accordance with the present invention, in which different examples of electrodes are employed, and

FIG. 15 is a schematic sectional view showing a modification of the first embodiment of the cleaning apparatus for a biochemical analysis unit in accordance with the present invention, in which a different example of a grounding system is employed.

### DESCRIPTION OF THE PREFERRED EMBODIMENTS

The present invention will hereinbelow be described in further detail with reference to the accompanying drawings.

In the assay method using a biochemical analysis unit in accordance with the present invention, a biochemical analysis unit provided with a plurality of porous adsorptive regions, to which ligands or receptors have been bound respectively, is utilized.

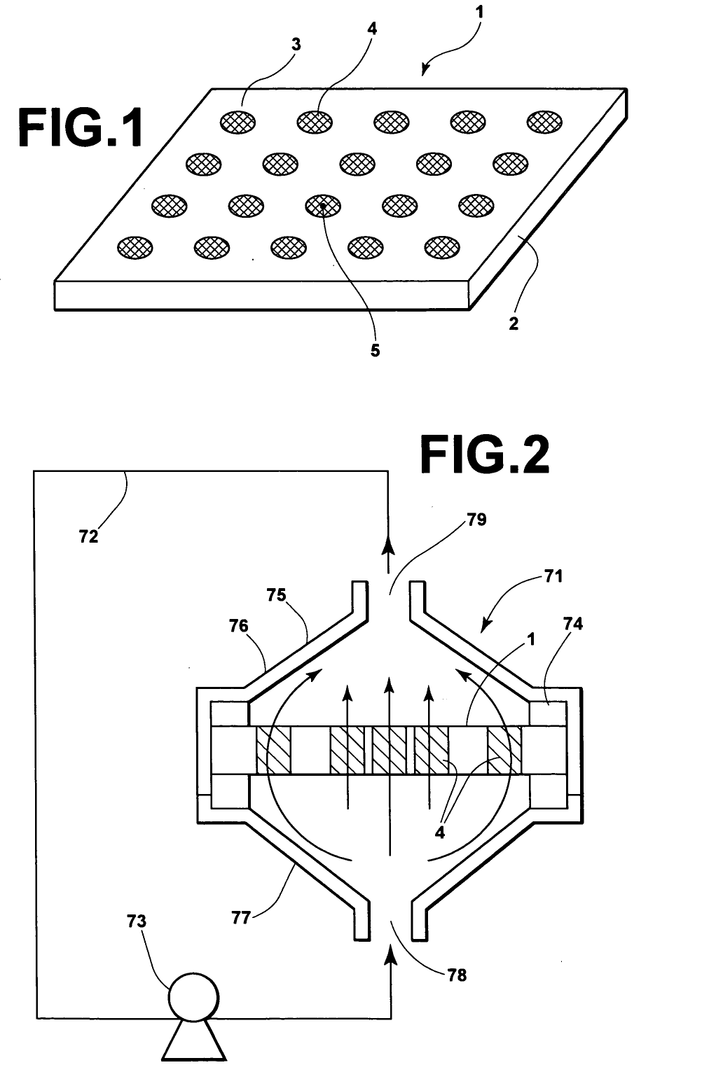

FIG. 1 is a schematic perspective view showing an example of a biochemical analysis unit utilized for the assay method using a biochemical analysis unit in accordance with the present invention. With reference to FIG. 1, a biochemical analysis unit 1 comprises a base plate 2, which is provided with a plurality of holes 3, 3, . . . , and a plurality of adsorptive regions 4, 4, each of which is filled in one of the holes 3, 3, . . . and comprises a porous material adhered to the base plate 2. Each of ligands or receptors, whose structures or characteristics are known, has been spotted onto one of the adsorptive regions 4, 4, . . . and has then been immobilized with treatment.

Such that light scattering may be prevented from occurring within the biochemical analysis unit 1, the base plate 2 should preferably be made from a material, which does not transmit light or which attenuates the light. The material for the formation of the base plate 2 should preferably be a metal or a ceramic material. Also, in cases where a plastic material, for which the hole making processing is capable of being performed easily, is employed as the material for the formation of the base plate 2, particles should preferably be dispersed within the plastic material, such that the light is capable of being attenuated even further.

Examples of the metals, which may be utilized preferably for the formation of the base plate 2, include copper, silver, gold, zinc, lead, aluminum, titanium, tin, chromium, iron, nickel, cobalt, tantalum, and alloys, such as stainless steel and bronze. Examples of the ceramic materials, which may be utilized preferably for the formation of the base plate 2, include alumina, zirconia, magnesia, and quartz. Examples of the plastic materials, which may be utilized preferably for the formation of the base plate 2, include polyolefins, such as a polyethylene and a polypropylene; polystyrenes; acrylic resins, such as a polymethyl methacrylate; polyvinyl chlorides; polyvinylidene chlorides; polyvinylidene fluorides; polytetrafluoroethylenes; polychlorotrifluoroethylenes; polycarbonates; polyesters, such as a polyethylene naphthalate and a polyethylene terephthalate; aliphatic polyamides, such as a 6-nylon and a 6,6-nylon; polyimides; polysulfones; polyphenylene sulfides; silicon resins, such as a polydiphenyl siloxane; phenolic resins, such as novolak; epoxy resins; polyurethanes; celluloses, such as cellulose acetate and nitrocellulose; copolymers, such as a butadiene-styrene copolymer; and blends of plastic materials.

Such that the density of the holes 3, 3, . . . made through the base plate 2 may be enhanced, the area (size) of the opening of each of the holes 3, 3, . . . may ordinarily be smaller than 5 mm2. The area of the opening of each of the holes 3, 3, . . . should preferably be smaller than 1 mm2, should more preferably be smaller than 0.3 mm2, and should most preferably be smaller than 0.01 mm2. Also, the area of the opening of each of the holes 3, 3, . . . , should preferably be at least 0.001 mm2.

The pitch of the holes 3, 3, . . . (i.e., the distance between the center points of two holes which are adjacent to each other) should preferably fall within the range of 0.05 mm to 3 mm. Also, the spacing between two adjacent holes 3, 3 (i.e., the shortest distance between edges of two adjacent holes 3, 3) should preferably fall within the range of 0.01 mm to 1.5 mm. The number (the array density) of the holes 3, 3, . . . may ordinarily be at least 10 holes/cm2. The number (the array density) of the holes 3, 3, . . . should preferably be at least 100 holes/cm2, should more preferably be at least 500 holes/cm2, and should most preferably be at least 1,000 holes/cm2. Also, the number (the array density) of the holes 3, 3, . . . should preferably be at most 100,000 holes/cm2, and should more preferably be at most 10,000 holes/cm2. The holes 3, 3, . . . need not necessarily be arrayed at equal spacing as illustrated in FIG. 1. For example, the holes 3, 3, . . . may be grouped into several number of blocks (units) comprising a plurality of holes and may be formed in units of the blocks.

In the assay method using a biochemical analysis unit in accordance with the present invention, as the porous material for the formation of the adsorptive regions of the biochemical analysis unit, a porous quality material or a fiber material may be utilized preferably. The porous quality material and the fiber material may be utilized in combination in order to form the adsorptive regions of the biochemical analysis unit. In the assay method using a biochemical analysis unit in accordance with the present invention, the porous material, which maybe utilized for the formation of the adsorptive regions of the biochemical analysis unit, may be an organic material, an inorganic material, or an organic-inorganic composite material.

The organic porous quality material, which may be utilized for the formation of the adsorptive regions of the biochemical analysis unit, may be selected from a wide variety of materials. However, the organic porous quality material should preferably be a carbon porous quality material, such as active carbon, or a porous quality material capable of forming a membrane filter. As the porous quality material capable of forming a membrane filter, a polymer soluble in a solvent should preferably be utilized. Examples of the polymers soluble in a solvent include cellulose derivatives, such as nitrocellulose, regenerated cellulose, cellulose acetate, and cellulose acetate butyrate; aliphatic polyamides, such as a 6-nylon, a 6,6-nylon, and a 4,10-nylon; polyolefins, such as a polyethylene and a polypropylene; chlorine-containing polymers, such as a polyvinyl chloride and a polyvinylidene chloride; fluorine resins, such as a polyvinylidene fluoride and a polytetrafluoride; polycarbonates; polysulfones; alginic acids and alginic acid derivatives, such as alginic acid, calcium alginate, and an alginic acid-polylysine polyion complex; and collagen. Copolymers or composite materials (mixture materials) of the above-enumerated polymers may also be utilized.

The fiber material, which may be utilized for the formation of the adsorptive regions of the biochemical analysis unit, may be selected from a wide variety of materials. Examples of the fiber materials, which may be utilized preferably, include the cellulose derivatives and the aliphatic polyamides enumerated above.

The inorganic porous quality material, which may be utilized for the formation of the adsorptive regions of the biochemical analysis unit, may be selected from a wide variety of materials. Examples of the inorganic porous quality materials, which may be utilized preferably, include metals, such as platinum, gold, iron, silver, nickel, and aluminum; oxides of metals, and the like, such as alumina, silica, titania, and zeolite; metal salts, such as hydroxyapatite and calcium sulfate; and composite materials of the above-enumerated materials.

Perforation of the plurality of the holes 3, 3, . . . through the base plate 2 may be performed with, for example, a punching technique for punching with a pin, a technique for electrical discharge machining, in which a pulsed high voltage is applied across electrodes in order to volatilize the base plate material, an etching technique, or a laser beam irradiation technique. In cases where the material of the base plate is a metal material or a plastic material, the biochemical analysis unit may be prepared with an operation for performing corona discharge or plasma discharge on the surface of the base plate, applying an adhesive agent to the surface of the base plate, and laminating the porous material for the formation of the adsorptive regions by use of means, such as a press. At the time of the lamination, the porous material for the formation of the adsorptive regions may be heated and softened, such that the adsorptive regions may be formed easily within the holes. Also, in cases where the porous material for the formation of the adsorptive regions is pressed against the base plate, the base plate and the porous material for the formation of the adsorptive regions may be divided previously into a plurality of sheets, and the plurality of the sheets may be pressed intermittently. Alternatively, a long web of the base plate and a long web of the porous material for the formation of the adsorptive regions may be conveyed continuously between two rolls.

In the assay method using a biochemical analysis unit in accordance with the present invention, the biochemical analysis unit having been prepared by use of the material and the technique described above may be utilized. Alternatively, a commercially available biochemical analysis unit may be utilized. It is also possible to utilize a biochemical analysis unit, in which the ligands or the receptors have already been bound respectively to the porous adsorptive regions. The assay method using a biochemical analysis unit in accordance with the present invention is also applicable to an array chip, which is recently available commercially and in which a capillary-like hollow base plate is utilized.

The assay method using a biochemical analysis unit in accordance with the present invention is applicable broadly to various assay processes for:
i) obtaining a biochemical analysis unit provided with a plurality of porous adsorptive regions, to which ligands or receptors have been bound respectively,
ii) subjecting at least one kind of a receptor or at least one kind of a ligand to specific binding with the ligands or the receptors, each of which has been bound to one of the porous adsorptive regions of the biochemical analysis unit, the receptor or the ligand being thereby specifically bound to at least one of the ligands, each of which has been bound to one of the porous adsorptive regions of the biochemical analysis unit, or at least one of the receptors, each of which has been bound to one of the porous adsorptive regions of the biochemical analysis unit, and
iii) detecting the receptor or the ligand, which has thus been specifically bound to at least one of the ligands or at least one of the receptors, by the utilization of a labeling substance.

In a first aspect, the assay method using a biochemical analysis unit in accordance with the present invention is applicable to an assay process for:
i) obtaining a biochemical analysis unit provided with a plurality of porous adsorptive regions, to which ligands or receptors have been bound respectively,
ii) subjecting at least one kind of a labeled receptor or at least one kind of a labeled ligand, which has been labeled with a labeling substance, to specific binding with the ligands or the receptors, each of which has been bound to one of the porous adsorptive regions of the biochemical analysis unit, the labeled receptor or the labeled ligand being thereby specifically bound to at least one of the ligands, each of which has been bound to one of the porous adsorptive regions of the biochemical analysis unit, or at least one of the receptors, each of which has been bound to one of the porous adsorptive regions of the biochemical analysis unit,
iii) performing a cleaning operation for removing the labeled receptor or the labeled ligand, which has been non-specifically bound to the ligands or the receptors having been bound respectively to the porous adsorptive regions of the biochemical analysis unit, and
iv) detecting the labeled receptor or the labeled ligand, which has thus been specifically bound to at least one of the ligands or at least one of the receptors.

In such cases, the labeled receptor or the labeled ligand is the substance, which has been sampled from an organism through extraction, isolation, or the like, or has been subjected to chemical treatment after being sampled, and which has been labeled with the labeling substance. The labeled receptor or the labeled ligand is capable of undergoing the specific binding with at least one of the ligands, each of which has been bound to one of the porous adsorptive regions of the biochemical analysis unit, or at least one of the receptors, each of which has been bound to one of the porous adsorptive regions of the biochemical analysis unit. Examples of the labeled receptors or the labeled ligands include hormones, tumor markers, enzymes, antibodies, antigens, abzymes, other proteins, nucleic acids, DNA's, and mRNA's.

Examples of the labeling substances include a radioactive labeling substance, a fluorescent labeling substance, and a labeling substance capable of producing the chemical luminescence when being brought into contact with a chemical luminescence substrate. The labeling substance may be a substance, which is capable of producing radiation by itself, a substance, which is capable of emitting light by itself, a substance, which is capable of forming a color by itself, or a substance, which is capable of producing fluorescence by itself when being exposed to light. Alternatively, the labeling substance may be a substance, which is capable of causing a chemical substance to emit light, to form a color, or to produce the fluorescence through, for example, decomposition or reaction of the chemical substance when being brought into contact with the chemical substance. As for the former type of the labeling substance, a radioactive isotope may be employed as the radiation producing labeling substance. Also, an acridinium ester, or the like, may be employed as the light emitting labeling substance. Further, gold colloidal particles, or the like, may be employed as the color forming labeling substance. Furthermore, fluorescein, or the like, may be employed as the fluorescent labeling substance. As the latter type of the labeling substance, an enzyme may be employed. Examples of the enzymes include alkaline phosphatase, peroxidase, luciferase, and β-galactosidase. When one of the above-enumerated enzymes acting as the labeling substance is brought into contact with a chemical luminescence substrate, a dye substrate, or a fluorescence substrate, the enzyme is capable of causing the chemical luminescence substrate to produce the chemical luminescence, causing the dye substrate to form a color, or causing the fluorescence substrate to produce the fluorescence.

By way of example, in cases where the enzyme is alkaline phosphatase, peroxidase, or luciferase, the chemical luminescence substrate maybe dioxetane, luminol, or luciferin, respectively. In cases where the enzyme is alkaline phosphatase, the dye substrate may be p-nitrophenyl phosphate. In cases where the enzyme is β-galactosidase, the dye substrate may be p-nitrophenyl-β-D-galactoside, or the like. In cases where the enzyme is alkaline phosphatase, the fluorescence substrate may be 4-methylumbellifer phosphoric acid. In cases where the enzyme is peroxidase, the fluorescence substrate may be 3-(4-hydroxyphenyl)-propionic acid. In cases where the enzyme is β-galactosidase, the fluorescence substrate may be 4-methylumbellifer-β-D-galactoside, or the like.

In a second aspect, the assay method using a biochemical analysis unit in accordance with the present invention is applicable to an assay process for:
i) obtaining a biochemical analysis unit provided with a plurality of porous adsorptive regions, to which ligands or receptors have been bound respectively,
ii) subjecting at least one kind of a receptor or at least one kind of a ligand to specific binding with the ligands or the receptors, each of which has been bound to one of the porous adsorptive regions of the biochemical analysis unit, the receptor or the ligand being thereby specifically bound to at least one of the ligands, each of which has been bound to one of the porous adsorptive regions of the biochemical analysis unit, or at least one of the receptors, each of which has been bound to one of the porous adsorptive regions of the biochemical analysis unit,
iii) performing a cleaning operation for removing the receptor or the ligand, which has been non-specifically bound to the ligands or the receptors having been bound respectively to the porous adsorptive regions of the biochemical analysis unit,
iv) subjecting a labeled body, which has been labeled with a labeling substance, to specific binding with the receptor or the ligand having been specifically bound to at least one of the ligands, each of which has been bound to one of the porous adsorptive regions of the biochemical analysis unit, or at least one of the receptors, each of which has been bound to one of the porous adsorptive regions of the biochemical analysis unit, and
v) detecting the receptor or the ligand, which has been specifically bound to at least one of the ligands or at least one of the receptors.

The aforesaid second aspect of the assay process is the so-called sandwich technique, wherein the receptor or the ligand, which is to be detected, is sandwiched between the ligand or the receptor, which has been bound to the adsorptive region, and the labeled body. In this case, the receptor or the ligand, which is to be detected, is the substance, which has been sampled from an organism through extraction, isolation, or the like, or has been subjected to chemical treatment after being sampled, and which has been labeled with the labeling substance. The receptor or the ligand is capable of undergoing the specific binding with at least one of the ligands, each of which has been bound to one of the porous adsorptive regions of the biochemical analysis unit, or at least one of the receptors, each of which has been bound to one of the porous adsorptive regions of the biochemical analysis unit. Examples of the receptors or the ligands, which are to be detected, include hormones, tumor markers, enzymes, antibodies, antigens, abzymes, other proteins, nucleic acids, DNA's, and mRNA's.

The labeled body, which has been labeled with the labeling substance, is a body, which has been labeled with the labeling substance described above and is capable of undergoing the specific binding with a reaction site of the receptor or the ligand, which is to be detected. Examples of the labeled bodies include antigens, antibodies, hormones, tumor markers, enzymes, abzymes, other proteins, nucleic acids, cDNA's, DNA's, and RNA's, whose characteristics, compositions, structures, base sequences, base lengths, and the like, are known.

In a third aspect, the assay method using a biochemical analysis unit in accordance with the present invention is applicable to an assay process for:
i) obtaining a biochemical analysis unit provided with a plurality of porous adsorptive regions, to which ligands or receptors have been bound respectively,
ii) subjecting at least one kind of an auxiliary substance-bound receptor or at least one kind of an auxiliary substance-bound ligand, to which an auxiliary substance has been bound, to specific binding with the ligands or the receptors, each of which has been bound to one of the porous adsorptive regions of the biochemical analysis unit, the auxiliary substance-bound receptor or the auxiliary substance-bound ligand being thereby specifically bound to at least one of the ligands, each of which has been bound to one of the porous adsorptive regions of the biochemical analysis unit, or at least one of the receptors, each of which has been bound to one of the porous adsorptive regions of the biochemical analysis unit,
iii) subjecting an auxiliary substance-combinable labeling substance, which is capable of undergoing specific binding with the auxiliary substance, to specific binding with the auxiliary substance of the auxiliary substance-bound receptor or the auxiliary substance-bound ligand having been specifically bound to at least one of the ligands, each of which has been bound to one of the porous adsorptive regions of the biochemical analysis unit, or at least one of the receptors, each of which has been bound to one of the porous adsorptive regions of the biochemical analysis unit, and
iv) detecting the auxiliary substance-bound receptor or the auxiliary substance-bound ligand, which has been specifically bound to at least one of the ligands or at least one of the receptors, by the utilization of the auxiliary substance-combinable labeling substance.

The auxiliary substance is a substance capable of undergoing the binding with the auxiliary substance-combinable labeling substance. Examples of preferable auxiliary substances include antigens, such as digoxigenin, biotin, avidin, and fluorescein, and antibodies with respect to the above-enumerated antigens. Also, the auxiliary substance may be a biological binding partner, such as avidin with respect to biotin. In this case, the auxiliary substance-combinable labeling substance is a substance, which is capable of undergoing the specific binding with the auxiliary substance and has been labeled with the labeling substance described above.

In a specific aspect, the assay method using a biochemical analysis unit in accordance with the present invention is applicable to an assay process for:
i) obtaining a biochemical analysis unit provided with a plurality of porous adsorptive regions, to which ligands or receptors have been bound respectively,
ii) subjecting at least one kind of an antigen-bound receptor or at least one kind of an antigen-bound ligand, to which an antigen has been bound, to specific binding with the ligands or the receptors, each of which has been bound to one of the porous adsorptive regions of the biochemical analysis unit, the antigen-bound receptor or the antigen-bound ligand being thereby specifically bound to at least one of the ligands, each of which has been bound to one of the porous adsorptive regions of the biochemical analysis unit, or at least one of the receptors, each of which has been bound to one of the porous adsorptive regions of the biochemical analysis unit,
iii) subjecting an enzyme-labeled antibody, which is an antibody with respect to the antigen having been bound to the antigen-bound receptor or the antigen-bound ligand and which has been labeled with an enzyme capable of producing chemical luminescence, to specific binding with the antigen of the antigen-bound receptor or the antigen-bound ligand having been specifically bound to at least one of the ligands, each of which has been bound to one of the porous adsorptive regions of the biochemical analysis unit, or at least one of the receptors, each of which has been bound to one of the porous adsorptive regions of the biochemical analysis unit, and
iv) detecting the antigen-bound receptor or the antigen-bound ligand, which has been specifically bound to at least one of the ligands or at least one of the receptors.

As illustrated in, for example, FIG. 2, in order to perform the specific binding of the receptor or the ligand with the ligands or the receptors, each of which has been bound to one of the adsorptive regions of the biochemical analysis unit, a reactor, in which a reaction liquid is capable of being forcibly caused to flow such that the reaction liquid flows across each of the adsorptive regions of the biochemical analysis unit, is utilized.

FIG. 2 is a schematic sectional view showing an example of a reactor, in which a reaction liquid is capable of being forcibly caused to flow. With reference to FIG. 2, the reactor comprises a reaction vessel 71, a liquid circulating pipe 72 and a pump 73. The reaction vessel 71 is provided with a biochemical analysis unit support section 74, which supports a biochemical analysis unit 1 and has sealing functions for preventing liquid leakage. A reaction vessel main body 75 of the reaction vessel 71 comprises a reaction vessel upper half 76 and a reaction vessel lower half 77. The reaction vessel upper half 76 is releasably secured to the reaction vessel main body 75. When the biochemical analysis unit 1 is to be set within the reaction vessel 71, the reaction vessel upper half 76 is dismounted from the reaction vessel main body 75, and the biochemical analysis unit 1 is set within the reaction vessel 71. A bottom wall of the reaction vessel lower half 77 is provided with a liquid inlet 78, through which a liquid is capable of flowing. Also, a top wall of the reaction vessel upper half 76 is provided with a liquid outlet 79, through which the liquid is capable of flowing. Further, the liquid circulating pipe 72 is releasably fitted to the liquid inlet 78 and the liquid outlet 79 of the reaction vessel 71. The reactor is constituted such that the liquid is introduced by the pump 73 into the reaction vessel main body 75 through the liquid inlet 78, passed through the biochemical analysis unit 1, discharged through the liquid outlet 79, and circulated through the liquid circulating pipe 72.

The biochemical analysis unit 1 provided with the adsorptive regions, to which the ligands or the receptors have been bound respectively, is set in the reactor. Also, the reaction liquid containing the receptor or the ligand is introduced into the reaction vessel 71. Thereafter, the pump 73 is actuated, and the reaction liquid is forcibly caused to flow such that the reaction liquid flows across each of the adsorptive regions of the biochemical analysis unit 1. In this manner, the receptor or the ligand is capable of being subjected to the specific binding with the ligands or the receptors, which have been bound respectively to the adsorptive regions of the biochemical analysis unit 1.

In the aforesaid example, the specific binding is performed by use of the reactor, which is capable of forcibly causing the reaction liquid to flow such that the reaction liquid flows across each of the adsorptive regions of the biochemical analysis unit. However, in the assay method using a biochemical analysis unit in accordance with the present invention, the specific binding is not limited to the use of the reactor described above. For example, the specific binding may be performed with the shaking technique, wherein the biochemical analysis unit and the reaction liquid are put into a reaction vessel, vibrations are given to the reaction vessel, and the receptor or the ligand is thus moved through convection or diffusion and is specifically bound to one of the ligands or the receptors having been fixed to the adsorptive regions of the biochemical analysis unit.

In the assay method using a biochemical analysis unit in accordance with the present invention, after the receptor or the ligand has been subjected to the specific binding with the ligands or the receptors, each of which has been bound to one of the adsorptive regions of the biochemical analysis unit, a cleaning operation is performed with a cleaning apparatus. In the cleaning apparatus, at least one electrode is located such that the electrode is capable of causing the electric current to flow across each of the adsorptive regions of the biochemical analysis unit, and a positive voltage is applied to the electrode in order to cause the electric current to flow across each of the porous adsorptive regions of the biochemical analysis unit. In this manner, the receptor or the ligand, which has been non-specifically bound to the ligands or the receptors having been bound respectively to the adsorptive regions of the biochemical analysis unit, is removed.

In a first aspect of the cleaning operation, in which at least one electrode is located such that the electrode is capable of causing the electric current to flow across each of the adsorptive regions of the biochemical analysis unit, a plurality of electrodes are located, such that each of the electrodes is associated with one of the plurality of the adsorptive regions of the biochemical analysis unit, the positive voltage is applied to each of the electrodes successively, and the electric current is thus caused to flow successively across each of the plurality of the adsorptive regions. The electric current is thus caused to flow ultimately across all of the plurality of the adsorptive regions.

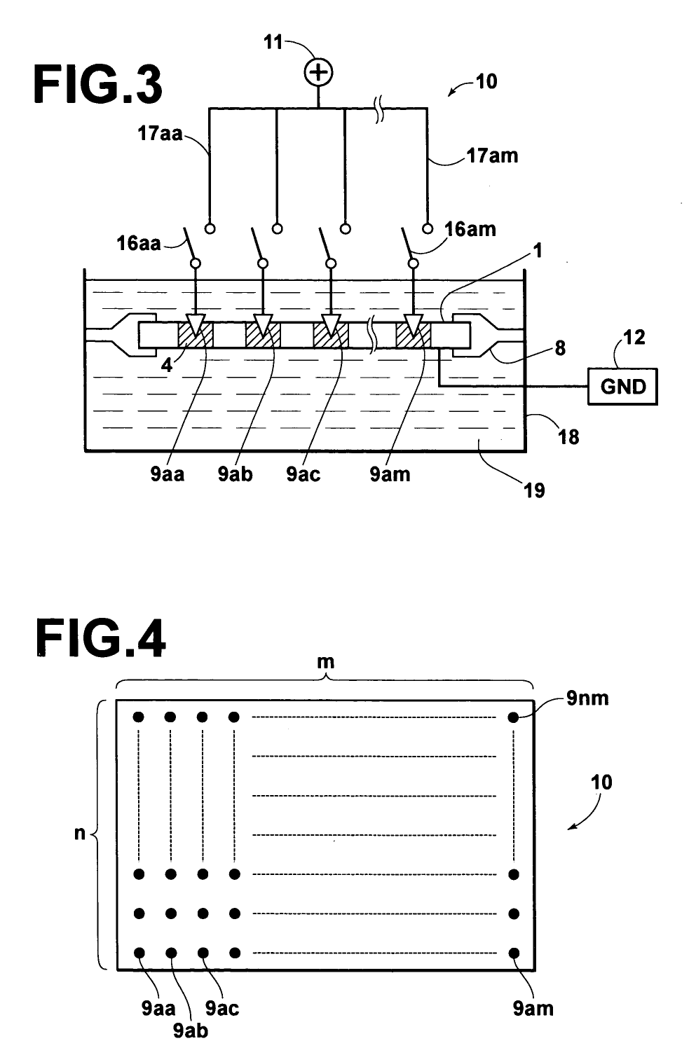

FIG. 3 is a schematic sectional view showing a first embodiment of the cleaning apparatus for a biochemical analysis unit in accordance with the present invention. FIG. 4 is a schematic bottom view showing an electric field forming device, which is employed in the first embodiment of the cleaning apparatus for a biochemical analysis unit in accordance with the present invention. As illustrated in FIG. 3, the first embodiment of the cleaning apparatus for a biochemical analysis unit in accordance with the present invention comprises a cleaning vessel 18, in which a cleaning liquid 19 is accommodated. The cleaning apparatus also comprises an electric field forming device 10, which acts as the control means. A biochemical analysis unit support section 8, which is capable of supporting the biochemical analysis unit 1, is formed within the cleaning vessel 18.

As illustrated in FIG. 4, the electric field forming device 10 comprises m×n number of electrodes 9aa, 9ab, 9ac, . . . , 9am, . . . , 9nm (nm=n×m). The electric field forming device 10 also comprises a positive electric power source 11. The electrodes 9aa, 9ab, 9ac, . . . , 9am, . . . , 9nm of the electric field forming device 10 are located, such that the position of each of the electrodes 9aa, 9ab, 9ac, . . . , 9am, . . . , 9nm corresponds to the position of one of the adsorptive regions 4, 4, . . . of the biochemical analysis unit 1. The electric field forming device 10 is capable of being moved by a motor (not shown) so as to take a position for electric field application, at which each of the electrodes 9aa, 9ab, 9ac, . . . , 9am, . . . , 9nm has been inserted into the corresponding one of the adsorptive regions 4, 4, . . . of the biochemical analysis unit 1 as illustrated in FIG. 3, and a retreat position, to which each of the electrodes 9aa, 9ab, 9ac, . . . , 9am, . . . , 9nm has retreated upwardly from the corresponding one of the adsorptive regions 4, 4, . . . of the biochemical analysis unit 1.

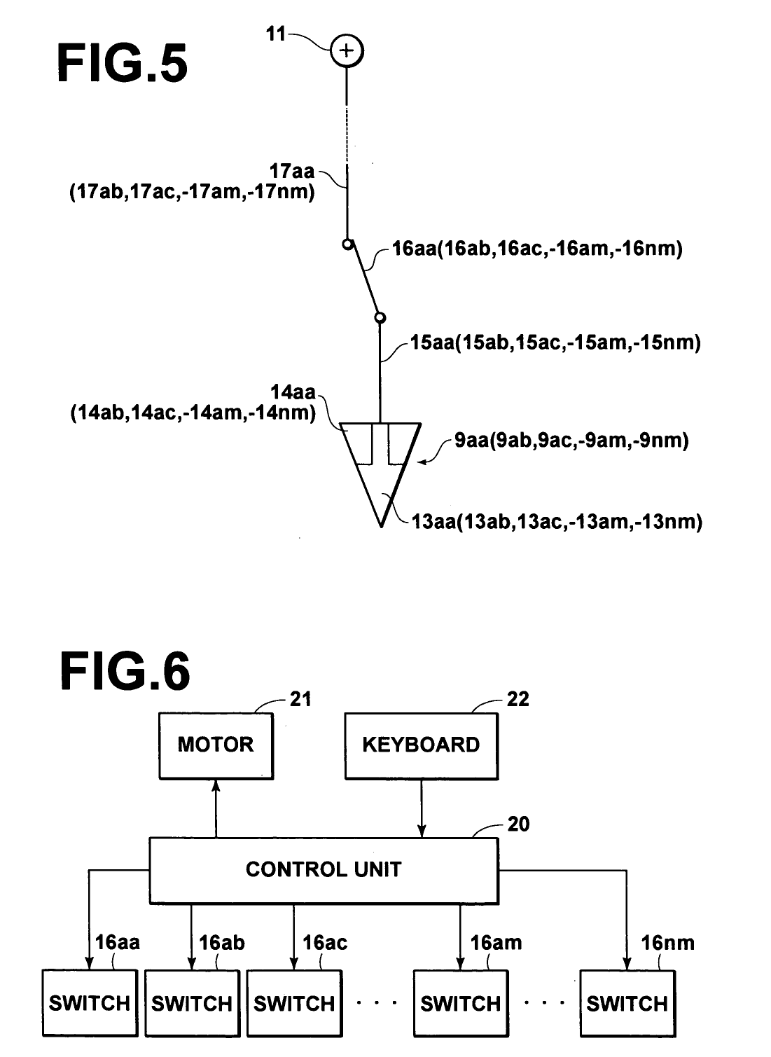

FIG. 5 is a schematic sectional view showing each of the electrodes 9aa, 9ab, 9ac, . . . , 9am, . . . , 9nm. As illustrated in FIG. 5, the electrode 9aa has a circular cone-like shape and comprises a pin-like electrical conductor 13aa and an electrical insulator 14aa, which covers a region of the electrical conductor 13aa other than a pointed end region of the electrical conductor 13aa. Each of the electrodes 9ab, 9ac, . . . , 9am, . . . , 9nm is constituted in the same manner as that for the electrode 9aa. In FIG. 5, reference numerals 13ab, 13ac, . . . , 13am, . . . , 13nm respectively represent electrical conductors of the electrodes 9ab, 9ac, . . . , 9am, . . . , 9nm. Also, reference numerals 14ab, 14ac, . . . , 14am, . . . , 14nm respectively represent electrical insulators of the electrodes 9ab, 9ac, . . . , 9am, . . . , 9nm. The electrical conductors 13aa, 13ab, 13ac, . . . , 13am, . . . , 13nm are electrically connected to conductor wires 15aa, 15ab, 15ac, . . . , 15am, . . . , 15nm, respectively. The conductor wires 15aa, 15ab, 15ac, . . . , 15am, . . . , 15nm are respectively connected to switches 16aa, 16ab, 16ac, . . . , 16am, . . . , 16nm. By the change-over of the settings of the switches 16aa, 16ab, 16ac, . . . , 16am, . . . , 16nm, the electrodes 9aa, 9ab, 9ac, . . . , 9am, . . . , 9nm are respectively connected to electrical conductors 17aa, 17ab, 17ac, . . . , 17am, . . . , 17nm, which are connected to the positive electric power source 11. As illustrated in FIG. 3, the biochemical analysis unit 1 is electrically connected to the ground 12.

FIG. 6 is a block diagram showing a control system, an actuating system, and an input system of the electric field forming device 10, which is employed in the first embodiment of the cleaning apparatus for a biochemical analysis unit in accordance with the present invention. As illustrated in FIG. 6, the control system of the electric field forming device 10 comprises a control unit 20 for controlling the operations of the entire cleaning apparatus. The control unit 20 is capable of performing on-off control of the positive electric power source 11 and control of the change-over of the settings of the switches 16aa, 16ab, 16ac, . . . , 16am, . . . , 16nm. The actuating system of the electric field forming device 10 comprises a motor 21 for moving the electric field forming device 10 between the position for electric field application and the retreat position. The input system of the electric field forming device 10 comprises a keyboard 22.

In the first embodiment of the cleaning apparatus for a biochemical analysis unit in accordance with the present invention, which has the constitution described above, firstly, the electric field forming device 10 is kept at the retreat position. Also, the biochemical analysis unit 1 provided with the adsorptive regions 4, 4, . . . , to which the ligands or the receptors have been bound, the receptor or the ligand having been specifically bound to at least one of the ligands or at least one of the receptors, is set at the biochemical analysis unit support section 8. The base plate 2 of the biochemical analysis unit 1 is made from an electrically conductive material, such that the electric current flows across each of the adsorptive regions 4, 4, . . . when positive electric charges are applied to the electrodes 9aa, 9ab, 9ac, . . . , 9am, . . . , 9nm.

After the cleaning liquid 19 has been accommodated within the cleaning vessel 18, a start signal is inputted from the keyboard 22. The start signal is fed into the control unit 20. The control unit 20 receives the start signal and feeds an actuation signal to the motor 21 in order to move the electric field forming device 10 from the retreat position to the position for electric field application. As a result, the circular cone-shaped electrodes 9aa, 9ab, 9ac, . . . , 9am, . . . , 9nm of the electric field forming device 10, each of which electrodes is located at the position corresponding to the position of one of the adsorptive regions 4, 4, . . . or the biochemical analysis unit 1, are inserted into the corresponding adsorptive regions 4, 4, . . . of the biochemical analysis unit 1.

Thereafter, the control unit 20 changes over the setting of the switch 16aa, which is connected to the electrode 9aa, such that the conductor wire 15aa and the conductor wire 17aa, which is connected to the positive electric power source 11, are electrically connected to each other. The electrode 9aa is thus electrically connected to the positive electric power source 11. After the electrode 9aa has thus been electrically connected to the positive electric power source 11, the control unit 20 turns on the positive electric power source 11. As a result, the positive voltage is applied to the electrode 9aa, and the electric current flows across the adsorptive region 4, into which the electrode 9aa has been inserted. Also, the receptor or the ligand, which has been non-specifically bound to the ligand or the receptor having been bound to the adsorptive region 4 of the biochemical analysis unit 1, is attracted toward the electrode 9aa. The receptor or the ligand, which has been non-specifically bound to the ligand or the receptor, thus separates from the adsorptive region 4, into which the electrode 9aa has been inserted, and moves to the surface of the electrode 9aa.

When a predetermined length of time has elapsed, the control unit 20 turns off the positive electric power source 11. When the positive electric power source 11 is turned off, the receptor or the ligand, which has been attracted to the surface of the electrode 9aa, separates from the surface of the electrode 9aa and moves into the cleaning liquid 19. In this manner, the receptor or the ligand, which has been non-specifically bound to the ligand or the receptor having been bound to the adsorptive region 4 of the biochemical analysis unit 1, is removed.

Thereafter, the control unit 20 changes over the setting of the switch 16aa and changes over the setting of the switch 16ab, which is connected to the electrode 9ab, such that the conductor wire 15ab and the conductor wire 17ab, which is connected to the positive electric power source 11, are electrically connected to each other. The electrode 9ab is thus electrically connected to the positive electric power source 11. After the electrode 9ab has thus been electrically connected to the positive electric power source 11, the control unit 20 turns on the positive electric power source 11. As a result, the positive voltage is applied to the electrode 9ab, and the electric current flows across the adsorptive region 4, into which the electrode 9ab has been inserted. Also, the receptor or the ligand, which has been non-specifically bound to the ligand or the receptor having been bound to the adsorptive region 4 of the biochemical analysis unit 1, is attracted toward the electrode 9ab. The receptor or the ligand, which has been non-specifically bound to the ligand or the receptor, thus separates from the adsorptive region 4, into which the electrode 9ab has been inserted, and moves to the surface of the electrode 9ab.

When a predetermined length of time has elapsed, the control unit 20 turns off the positive electric power source 11. When the positive electric power source 11 is turned off, the receptor or the ligand, which has been attracted to the surface of the electrode 9ab, separates from the surface of the electrode 9ab and moves into the cleaning liquid 19. In this manner, the receptor or the ligand, which has been non-specifically bound to the ligand or the receptor having been bound to the adsorptive region 4 of the biochemical analysis unit 1, is removed.

In the manner described above, the settings of the switches 16aa, 16ab, 16ac, . . . , 16am, . . . , 16nm are successively changed over, and an electrode 9jk (where j=a, . . . , n, and k=a, . . . , m), which is an electrode among the electrodes 9aa, 9ab, 9ac, . . . , 9am, . . . , 9nm of the electric field forming device 10, is electrically connected to the positive electric power source 11. The positive voltage is thus applied to the electrode 9jk, and the electric current is caused to flow across the adsorptive region 4, into which the electrode 9jk has been inserted. As a result, the receptor or the ligand, which has been non-specifically bound to the ligand or the receptor having been bound to the adsorptive region 4 of the biochemical analysis unit 1, and which has been negatively charged, is attracted toward the electrode 9jk. The receptor or the ligand, which has been non-specifically bound to the ligand or the receptor, thus separates from the adsorptive region 4, into which the electrode 9jk has been inserted, and moves to the surface of the electrode 9jk.

When a predetermined length of time has elapsed, the control unit 20 turns off the positive electric power source 11. When the positive electric power source 11 is turned off, the receptor or the ligand, which has been attracted to the surface of the electrode 9jk, separates from the surface of the electrode 9jk and moves into the cleaning liquid 19. In this manner, the receptor or the ligand, which has been non-specifically bound to the ligand or the receptor having been bound to the adsorptive region 4 of the biochemical analysis unit 1, is removed.

The ligands or the receptors, which have been bound respectively to the plurality of the adsorptive regions 4, 4, . . . of the biochemical analysis unit 1, vary in structure, and therefore vary in state of non-specific binding. Therefore, in accordance with a presumed state of the non-specific binding, the value of the positive voltage applied to the electrode 9jk should preferably be altered for each of the adsorptive regions 4, 4, . . . The voltage applied to the electrode 9jk is adjusted such that the receptor or the ligand, which has been perfectly bound through the specific binding to the ligand or the receptor having been bound to the adsorptive region 4, does not separate from the ligand or the receptor, and such that the receptor or the ligand, which has been non-specifically bound to the ligand or the receptor having been bound to the adsorptive region 4, separates from the ligand or the receptor. As described above, the biochemical analysis unit 1 is electrically connected to the ground 12. Therefore, in cases where the positive voltage is applied to the electrode 9jk in order to cause the electric current to flow across the adsorptive region 4, into which the electrode 9jk has been inserted, adverse effects of the voltage in the adsorptive region 4 associated with the electrode 9jk, to which the positive voltage is applied, upon the adsorptive region 4 associated with the electrode, to which the positive voltage is not applied, are capable of being minimized.

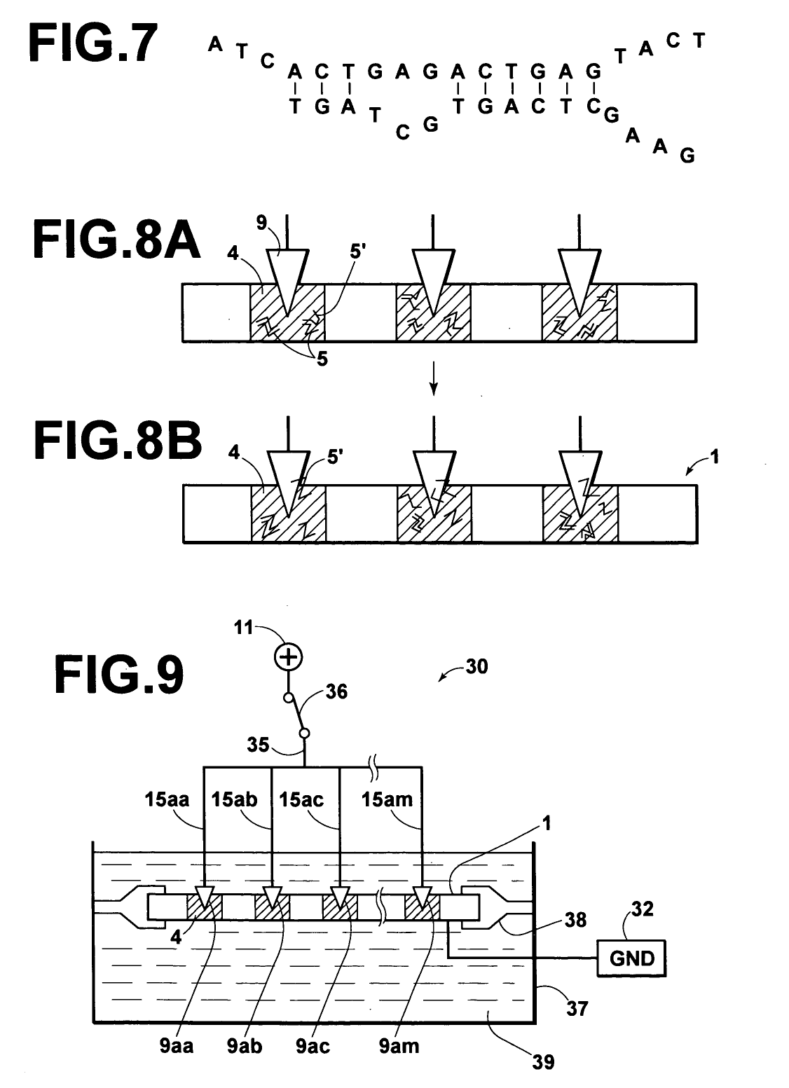

FIG. 7 is an explanatory view showing how cross hybridization of DNA's occurs. In the cases of the DNA's, adenine (A) and thymine (T) complementarily bind to each other through the formation of a base pair. Also, cytonsine (C) and guanine (G) complementarily bind to each other through the formation of a base pair. However, in the cases of sequences similar to each other, even though a site, at which the complementary binding does not occur, is present partially, the hybridization occurs. The receptor or the ligand, part of which has not undergone the complementary binding, partially undergoes the complementary binding to the ligand or the receptor having been bound to the adsorptive region, and therefore is not always capable of being removed with the ordinary washing operation, which is performed in the ordinary assay processes in order to remove an unreacted receptor or an unreacted ligand remaining in an adsorptive region.

Also, in the aforesaid assay method, wherein the auxiliary substance-combinable labeling substance is subjected to the specific binding with the auxiliary substance of the auxiliary substance-bound receptor or the auxiliary substance-bound ligand having been specifically bound to at least one of the ligands or at least one of the receptors, it may occur that the auxiliary substance-combinable labeling substance, such as the enzyme-labeled antibody, is non-specifically bound to the auxiliary substance-bound receptor or the auxiliary substance-bound ligand. Also, it may occur that the auxiliary substance-combinable labeling substance, such as the enzyme-labeled antibody, is not bound to the auxiliary substance-bound receptor or the auxiliary substance-bound ligand and remains in the adsorptive region. Ordinarily, the enzyme-labeled antibody has negative electric charges. The enzyme-labeled antibody, which has undergone the non-specific binding, is not always capable of being removed with the ordinary washing operation utilizing a washing liquid. Further, it may often occur that the enzyme-labeled antibody, which has not been bound to the auxiliary substance-bound receptor or the auxiliary substance-bound ligand and remains in the adsorptive region, is apt to cling to the adsorptive region and is not capable of being removed perfectly with the ordinary washing operation. However, in cases where the electric field is applied to the adsorptive region, the surplus enzyme-labeled antibody, the enzyme-labeled antibody which has undergone the non-specific binding and is not always capable of being removed with the ordinary washing operation, and the enzyme-labeled antibody which clings to the adsorptive region, are capable of being removed.

FIG. 8A is an explanatory view showing how a receptor or a ligand is non-specifically bound to ligands or receptors, each of which has been bound to one of porous adsorptive regions of the biochemical analysis unit. FIG. 8B is an explanatory view showing how the receptor or the ligand having been non-specifically bound to the ligands or the receptors, each of which has been bound to one of porous adsorptive regions of the biochemical analysis unit, separates from the ligands or the receptors. As illustrated in FIG. 8A, a ligand 5 or a receptor 5 has been bound to the adsorptive region 4 of the biochemical analysis unit 1, and a receptor or a ligand has been specifically bound to the ligand 5 or the receptor 5. However, it may occur that a receptor 5′ or a ligand 5′, which does not have the characteristics of undergoing the specific binding to the ligand 5 or the receptor 5, is bound to only part of the ligand 5 or the receptor 5 having been bound to the adsorptive region 4. As illustrated in FIG. 8B, in cases where the positive voltage is applied to the electrode 9, and the electric current flows across the adsorptive region 4, into which the electrode 9 has been inserted, the receptor 5′ or the ligand 5′, which has been non-specifically bound to the ligand 5 or the receptor 5 and has been charged negatively, is attracted toward the electrode 9, separates from the adsorptive region 4, and moves to the surface of the electrode 9. The receptor 5′ or the ligand 5′, which has been non-specifically bound to the ligand 5 or the receptor 5, is thus capable of being removed from the adsorptive region 4. In the same manner, the aforesaid enzyme-labeled antibody, which has been charged negatively, is capable of being removed from the adsorptive region 4.

In the first embodiment of the cleaning apparatus for a biochemical analysis unit in accordance with the present invention, the settings of the switches 16aa, 16ab, 16ac, . . . , 16am, . . . , 16nm of the electric field forming device 10 are successively changed over, and the electrode 9jk (where j=a, . . . , n, and k=a, . . . , m), which is an electrode among the electrodes 9aa, 9ab, 9ac, . . . , 9am, . . . , 9nm of the electric field forming device 10, is electrically connected to the positive electric power source 11. The positive voltage is thus applied to the electrode 9jk, which is connected to the positive electric power source 11, and the electric current is caused to flow across the adsorptive region 4, into which the electrode 9jk has been inserted. In this manner, the cleaning operation is performed. Alternatively, groups of at least two electrodes may be successively connected to the positive electric power source 11, the positive voltage may be applied to the group of the at least two electrodes, which are connected to the positive electric power source 11, and the cleaning operation may thus be performed. As another alternative, the settings of the switches 16aa, 16ab, 16ac, . . . , 16am, . . . , 16nm of the electric field forming device 10 may be successively changed over, such that the plurality of the electrode rows are successively connected to the positive electric power source 11, and such that the positive voltage is applied to the electrodes constituting the electrode row connected to the positive electric power source 11 in order to cause the electric current to flow across each of the adsorptive regions 4, 4, . . . , into which the electrodes constituting the electrode row have been inserted. The cleaning operation may thus be performed.

In a second aspect of the cleaning operation, in which at least one electrode is located such that the electrode is capable of causing the electric current to flow across each of the adsorptive regions of the biochemical analysis unit, a plurality of electrodes are located, such that each of the electrodes is associated with one of the plurality of the adsorptive regions of the biochemical analysis unit, the positive voltage is applied to all of the electrodes simultaneously, and the electric current is thus caused to flow simultaneously across each of the plurality of the adsorptive regions.

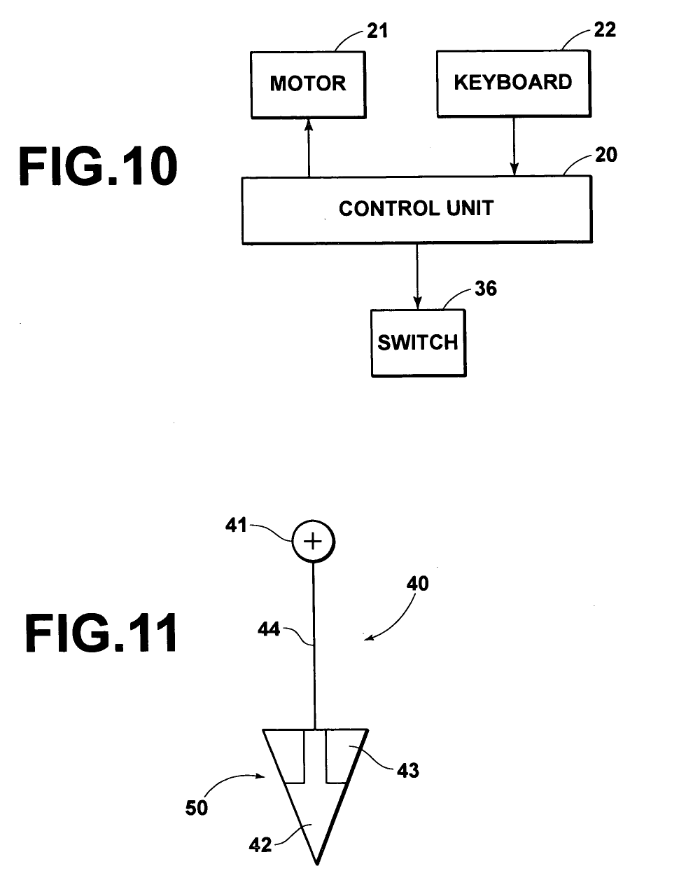

FIG. 9 is a schematic sectional view showing a second embodiment of the cleaning apparatus for a biochemical analysis unit in accordance with the present invention. FIG. 10 is a block diagram showing a control system, an actuating system, and an input system of an electric field forming device, which is employed in the second embodiment of the cleaning apparatus for a biochemical analysis unit in accordance with the present invention. As illustrated in FIG. 9, the second embodiment of the cleaning apparatus for a biochemical analysis unit in accordance with the present invention comprises an electric field forming device 30. As in the first embodiment of the cleaning apparatus for a biochemical analysis unit in accordance with the present invention, the electric field forming device 30 comprises the m×n number of the electrodes 9aa, 9ab, 9ac, . . . , 9am, . . . , 9nm (nm=n×m) and the positive electric power source 11. As illustrated in FIG. 9, in the second embodiment of the cleaning apparatus for a biochemical analysis unit in accordance with the present invention, the conductor wires 15aa, 15ab, 15ac, . . . , 15am, . . . , 15nm, which are respectively connected to the electrodes 9aa, 9ab, 9ac, . . . , 9am, . . . , 9nm of the electric field forming device 30, are electrically connected to a conductor wire 35. The conductor wire 35 is electrically connected to a switch 36. The electrodes 9aa, 9ab, 9ac, . . . , 9am, . . . , 9nm are thus capable of being simultaneously connected to the positive electric power source 11.

Therefore, as illustrated in FIG. 10, the control unit 20 is constituted so as to change over the setting of the switch 36 alone. In the second embodiment of the cleaning apparatus for a biochemical analysis unit in accordance with the present invention, firstly, the biochemical analysis unit 1 provided with the adsorptive regions 4, 4, . . . , to which the ligands or the receptors have been bound, the receptor or the ligand having been specifically bound to at least one of the ligands or at least one of the receptors, is set at a biochemical analysis unit support section 38. The base plate 2 of the biochemical analysis unit 1 is made from an electrically conductive material, such that the electric current flows across each of the adsorptive regions 4, 4, . . . when positive electric charges are applied to the electrodes 9aa, 9ab, 9ac, . . . , 9am, . . . , 9nm.

After a cleaning liquid 39 has been accommodated within the cleaning vessel 37, a start signal is inputted from the keyboard 22. The start signal is fed into the control unit 20. The control unit 20 receives the start signal and feeds an actuation signal to the motor 21 in order to move the electric field forming device 30 from the retreat position to the position for electric field application. As a result, the circular cone-shaped electrodes 9aa, 9ab, 9ac, . . . , 9am, . . . , 9nm of the electric field forming device 30, each of which electrodes is located at the position corresponding to the position of one of the adsorptive regions 4, 4, . . . or the biochemical analysis unit 1, are inserted into the corresponding adsorptive regions 4, 4, . . . of the biochemical analysis unit 1.

Thereafter, the control unit 20 changes over the setting of the switch 36, such that the conductor wire 35 and the positive electric power source 11 are electrically connected to each other. The electrodes 9aa, 9ab, 9ac, . . . , 9am, . . . , 9nm are thus electrically connected to the positive electric power source 11. After the setting of the switch 36 has thus been changed over, and the electrodes 9aa, 9ab, 9ac, . . . , 9am, . . . , 9nm have thus been electrically connected to the positive electric power source 11, the control unit 20 turns on the positive electric power source 11.

As a result, the positive voltage is applied to all of the electrodes 9aa, 9ab, 9ac, . . . , 9am, . . . , 9nm simultaneously, and the electric current flows across each of the adsorptive regions 4, 4, . . . , into which the electrodes 9aa, 9ab, 9ac, . . . , 9am, . . . , 9nm have respectively been inserted. Also, the receptor or the ligand, which has been non-specifically bound to the ligand or the receptor having been bound to each of the adsorptive regions 4, 4, . . . of the biochemical analysis unit 1, is attracted toward the corresponding one of the electrodes 9aa, 9ab, 9ac, . . . , 9am, . . . , 9nm. The receptor or the ligand, which has been non-specifically bound to the ligand or the receptor, thus separates from each of the adsorptive regions 4, 4, . . . into which the electrodes 9aa, 9ab, 9ac, . . . , 9am, . . . , 9nm have respectively been inserted, and moves to the surface of the corresponding one of the electrodes 9aa, 9ab, 9ac, . . . , 9am, . . . , 9nm.

When a predetermined length of time has elapsed, the control unit 20 turns off the positive electric power source 11. When the positive electric power source 11 is turned off, the receptor or the ligand, which has been attracted to the surface of the corresponding one of the electrodes 9aa, 9ab, 9ac, . . . , 9am, . . . , 9nm, separates from the surface of the corresponding electrode and moves into the cleaning liquid 39. In this manner, the receptor or the ligand, which has been non-specifically bound to the ligands or the receptors having been bound to the adsorptive regions 4, 4, . . . of the biochemical analysis unit 1, is removed.

In a third aspect of the cleaning operation, in which at least one electrode is located such that the electrode is capable of causing the electric current to flow across each of the adsorptive regions of the biochemical analysis unit, only one electrode is utilized and moved to each of the plurality of the adsorptive regions successively. Also, the positive voltage is applied to the electrode, and the electric current is thus caused to flow successively across each of the plurality of the adsorptive regions.

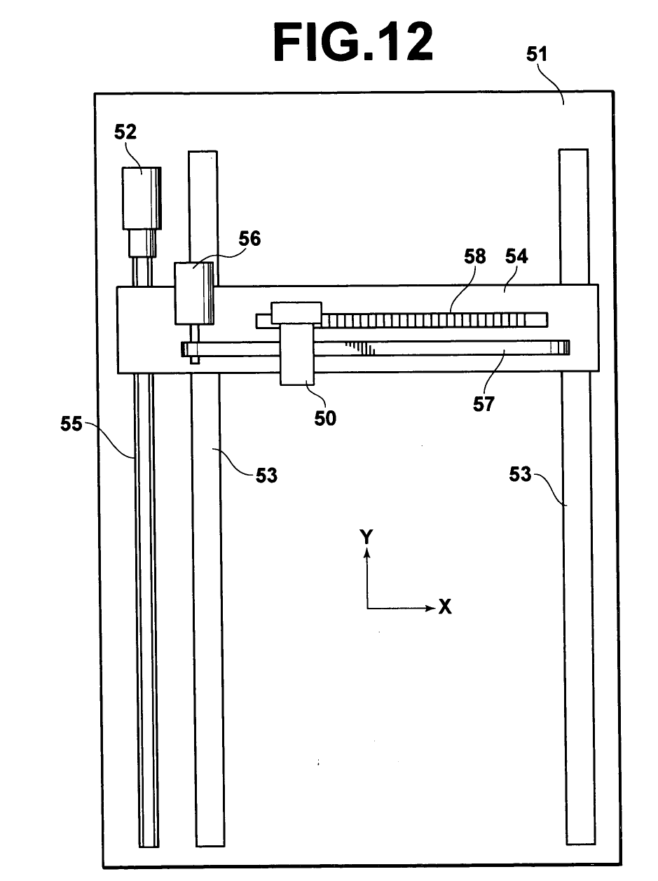

FIG. 11 is a schematic sectional view showing an electrode of an electric field forming device, which is employed in a third embodiment of the cleaning apparatus for a biochemical analysis unit in accordance with the present invention. FIG. 12 is a schematic plan view showing the third embodiment of the cleaning apparatus for a biochemical analysis unit in accordance with the present invention. As illustrated in FIG. 11, the third embodiment of the cleaning apparatus for a biochemical analysis unit in accordance with the present invention comprises an electric field forming device 40, which is provided with a single electrode 50. The electrode 50 comprises a pin-like electrical conductor 42 and an electrical insulator 43, which covers a region of the electrical conductor 42 other than a pointed end region of the electrical conductor 42. The electrical conductor 42 of the electrode 50 is electrically connected to a conductor wire 44, which is electrically connected to a positive electric power source 41.

As illustrated in FIG. 12, in the third embodiment of the cleaning apparatus for a biochemical analysis unit in accordance with the present invention, the electrode 50 of the electric field forming device 40 is capable of being moved in a main scanning direction, which is indicated by the arrow X, and a sub-scanning direction, which is indicated by the arrow Y. An actuating mechanism of the electric field forming device 40 is secured to a frame 51, which is secured to the vessel of the cleaning apparatus. A sub-scanning pulse motor 52 and a pair of rails 53, 53 are secured to the frame 51. Also, a plate 54 is located on the frame 51. The plate 54 is capable of being moved in the sub-scanning direction, which is indicated by the arrow Y, and along the pair of the rails 53, 53. The plate 54 has a threaded hole (not shown), and a threaded rod 55, which is rotated by the sub-scanning pulse motor 52, is engaged with the threaded hole of the plate 54. A main scanning pulse motor 56 is located on the plate 54. The main scanning pulse motor 56 is capable of intermittently rotating an endless belt 57 at predetermined pitches. Further, a linear encoder 58 for detecting the position of the electrode 50 with respect to the main scanning direction is located in parallel with the endless belt 57.

The electrode 50 is fitted to the endless belt 57, such that the electrode 50 is capable of being moved upwardly and downwardly by a solenoid (not shown in FIG. 12). When the endless belt 57 is rotated by the main scanning pulse motor 56, the electrode 50 is moved in the main scanning direction, which is indicated by the arrow X in FIG. 12.

FIG. 13 is a block diagram showing a control system, an actuating system, an input system, and a detection system of an electric field forming device, which is employed in the third embodiment of the cleaning apparatus for a biochemical analysis unit in accordance with the present invention. The electric field forming device 40 comprises a control unit 60, which acts as the control system and controls the operations of the entire electric field forming device 40. The electric field forming device 40 also comprises a keyboard 62, which acts as the input system. The control unit 60 is capable of performing on-off control of the positive electric power source 41. The actuating system of the electric field forming device 40 comprises the main scanning pulse motor 56, the sub-scanning pulse motor 52, and a solenoid 63 for vertically moving the electrode 50. The detection system of the electric field forming device 40 comprises the linear encoder 58 for detecting the position of the electrode 50 with respect to the main scanning direction, and a rotary encoder 67 for detecting the rotation quantity of the threaded rod 55.

In the third embodiment of the cleaning apparatus for a biochemical analysis unit in accordance with the present invention, as in the first and second embodiments of the cleaning apparatus in accordance with the present invention, the biochemical analysis unit 1 is set within the cleaning vessel, and a cleaning liquid is accommodated within the cleaning vessel. Thereafter, information representing the positions of the adsorptive regions 4, 4, . . . of the biochemical analysis unit 1 is inputted from the keyboard 62. The position information having been inputted from the keyboard 62 is fed into the control unit 60. The control unit 60 receives the position information and calculates actuation pulses, which are to be given to the main scanning pulse motor 56 and the sub-scanning pulse motor 52 in order to move the electrode 50 to the positions of the adsorptive regions 4, 4, . . . of the biochemical analysis unit 1. The calculated actuation pulse data are stored in a memory (not shown). When the actuation pulse data have been stored in the memory, a start signal is inputted from the keyboard 62.

The start signal is fed into the control unit 60. The control unit 60 receives the start signal and gives predetermined actuation pulses to the main scanning pulse motor 56 and the sub-scanning pulse motor 52 in accordance with the actuation pulse data, which have been stored in the memory, in order to move the electrode 50. At the time at which the electrode 50 arrives at a position, which stands facing a first adsorptive region 4 of the biochemical analysis unit 1, the control unit 60 feeds out actuation ceasing signals into the main scanning pulse motor 56 and the sub-scanning pulse motor 52 in order to stop the electrode 50 at the position described above. Also, the control unit 60 feeds out an actuation signal into the solenoid 63 in order to move the electrode 50 downwardly. The electrode 50 is thus inserted into the first adsorptive region 4 of the biochemical analysis unit 1. Thereafter, the control unit 60 turns on the positive electric power source 41. As a result, the positive voltage is applied to the electrode 50, and the electric current flows across the adsorptive region 4, into which the electrode 50 has been inserted. Also, the receptor or the ligand, which has been non-specifically bound to the ligand or the receptor having been bound to the adsorptive region 4 of the biochemical analysis unit 1, is attracted toward the electrode 50. The receptor or the ligand, which has been non-specifically bound to the ligand or the receptor, thus separates from the adsorptive region 4, into which the electrode 50 has been inserted, and moves to the surface of the electrode 50.

When a predetermined length of time has elapsed, the control unit 60 turns off the positive electric power source 41. Also, the control unit 60 feeds out an actuation ceasing signal into the solenoid 63. The electrode 50 is thus moved upwardly and retreated from the first adsorptive region 4 of the biochemical analysis unit 1.

Thereafter, the control unit 60 gives predetermined actuation pulses to the main scanning pulse motor 56 and the sub-scanning pulse motor 52 in accordance with the actuation pulse data, which have been stored in the memory, in order to move the electrode 50 to a position, which stands facing a second adsorptive region 4 of the biochemical analysis unit 1. At the time at which the electrode 50 arrives at the position, which stands facing the second adsorptive region 4 of the biochemical analysis unit 1, the control unit 60 feeds out actuation ceasing signals into the main scanning pulse motor 56 and the sub-scanning pulse motor 52 in order to stop the electrode 50 at the position described above. Also, the control unit 60 feeds out the actuation signal into the solenoid 63 in order to move the electrode 50 downwardly. The electrode 50 is thus inserted into the second adsorptive region 4 of the biochemical analysis unit 1. Thereafter, the control unit 60 turns on the positive electric power source 41. As a result, the positive voltage is applied to the electrode 50, and the electric current flows across the adsorptive region 4, into which the electrode 50 has been inserted. Also, the receptor or the ligand, which has been non-specifically bound to the ligand or the receptor having been bound to the adsorptive region 4 of the biochemical analysis unit 1, is attracted toward the electrode 50. The receptor or the ligand, which has been non-specifically bound to the ligand or the receptor, thus separates from the adsorptive region 4, into which the electrode 50 has been inserted, and moves to the surface of the electrode 50.

When a predetermined length of time has elapsed, the control unit 60 turns off the positive electric power source 41. Also, the control unit 60 feeds out the actuation ceasing signal into the solenoid 63. The electrode 50 is thus moved upwardly and retreated from the second adsorptive region 4 of the biochemical analysis unit 1.

In the same manner as that described above, by the main scanning pulse motor 56 and the sub-scanning pulse motor 52, the electrode 50 is moved at predetermined pitches in the main scanning direction, which is indicated by the arrow X, and the sub-scanning direction, which is indicated by the arrow Y. The electrode 50 is thus successively inserted into the adsorptive regions 4, 4, . . . , of the biochemical analysis unit 1, and the cleaning operation is performed.

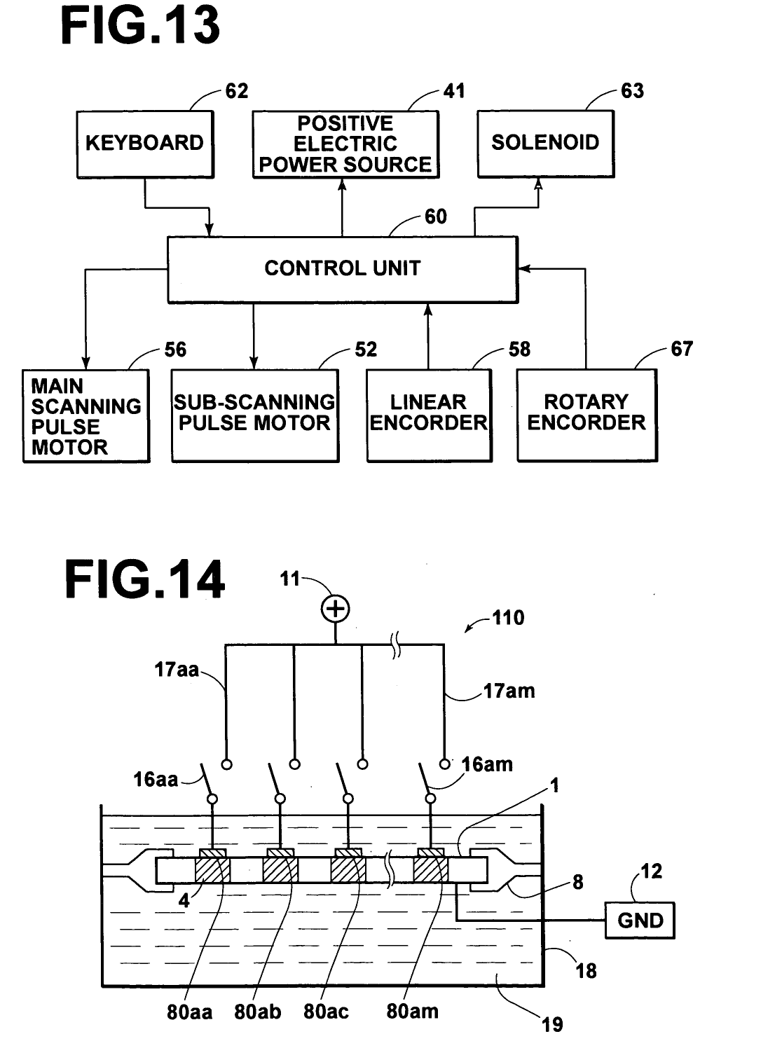

In each of the first, second, and third embodiments of the cleaning apparatus for a biochemical analysis unit in accordance with the present invention, the electrode having the circular cone-like shape is inserted into the adsorptive region 4, and the voltage is applied to the electrode in order to cause the electric current to flow across the adsorptive region 4. However, the shape of the electrode need not necessarily be the circular cone-like shape. Also, the electrode need not necessarily be inserted into the adsorptive region 4 and may be associated with the adsorptive region 4 in one of various other ways, such that the electrode is capable of causing the electric current to flow across the adsorptive region 4. For example, as in an electric field forming device 110 illustrated in FIG. 14, each of electrodes 80aa, 80ab, 80ac, . . . , 80am, which have flat circular disk-like shapes, may be brought into contact with the surface of one of the adsorptive regions 4, 4, . . . . As another alternative, instead of being brought into contact with the surface of one of the adsorptive regions 4, 4, . . . , each of electrodes 80aa, 80ab, 80ac, . . . , 80am, which have the flat circular disk-like shapes, may be located in the vicinity of the surface of one of the adsorptive regions 4, 4, . . . .

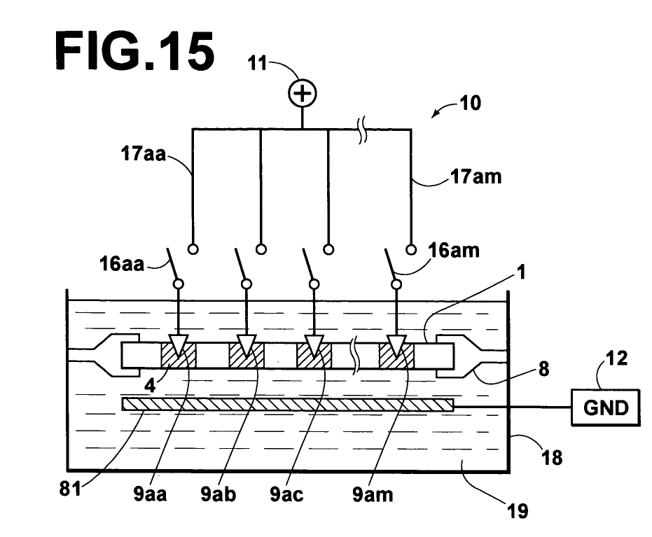

Also, in each of the first, second, and third embodiments of the cleaning apparatus for a biochemical analysis unit in accordance with the present invention, the base plate 2 of the biochemical analysis unit 1 is constituted of the electrically conductive material. However, the base plate 2 of the biochemical analysis unit 1 need not necessarily be constituted of the electrically conductive material. For example, as illustrated in FIG. 15, an electrically conductive plate 81 may be located on the side of the bottom surface of the cleaning vessel 18 of the cleaning apparatus and may be connected to the ground 12. With the constitution of the cleaning apparatus illustrated in FIG. 15, in cases where the base plate 2 of the biochemical analysis unit 1 is constituted of a material, which does not have the electrically conductive properties, the electric current is capable of being caused to flow across the adsorptive region 4.

Further, in the assay method using a biochemical analysis unit in accordance with the present invention, at the time of the cleaning operation for removing the receptor or the ligand, which has been non-specifically bound to the ligand or the receptor, at least one electrode is located such that the electrode is capable of causing the electric current to flow across each of the porous adsorptive regions of the biochemical analysis unit, and the positive voltage is applied to the electrode in order to cause the electric current to flow across each of the porous adsorptive regions of the biochemical analysis unit. The receptor or the ligand, which has been non-specifically bound to the adsorptive region, is thus removed from the adsorptive region. The technique for utilizing the electrode in the assay method using a biochemical analysis unit in accordance with the present invention may be utilized at a stage, at which a reaction liquid containing the receptor or the ligand capable of undergoing the specific binding with at least one of the ligands or the receptors having been bound to the adsorptive regions is introduced into the cleaning vessel of the cleaning apparatus, and the receptor or the ligand is thus subjected to the specific binding with the ligands or the receptors having been bound to the adsorptive regions. At this stage, at least one electrode may be located such that the electrode is capable of causing the electric current to flow across each of the adsorptive regions of the biochemical analysis unit, and the positive voltage may be applied to the electrode in order to cause the electric current to flow across each of the adsorptive regions of the biochemical analysis unit. In such cases, the receptor or the ligand, which is contained in the reaction liquid, is attracted toward the electrode and is thus forcibly brought into contact with the ligand or the receptor, which has been bound to each of the adsorptive regions. Therefore, the receptor or the ligand is capable of being subjected to the specific binding with the ligand or the receptor, which has been bound to each of the adsorptive regions.

In the embodiments described above, the positive voltage is applied to the electrode. However, the voltage is not limited to the positive voltage. Specifically, in cases where the non-specifically bound substance has positive electric charges, a negative voltage should preferably be applied to the electrode. Also, in the embodiments described above, the voltage is applied to the electrode in the state in which the cleaning liquid has been accommodated within the cleaning vessel. Alternatively, in cases where the adsorptive regions contain a sufficient amount of a liquid, the application of the voltage to the electrode may be performed without the cleaning liquid being used. In such cases, the operation for retreating the electrode from the adsorptive region should preferably be performed in the state in which the voltage is applied to the electrode.

1. An assay method using a biochemical analysis unit, comprising the steps of:
i) obtaining a biochemical analysis unit provided with a plurality of porous adsorptive regions, to which ligands or receptors have been bound respectively,
ii) subjecting at least one kind of a receptor or at least one kind of a ligand to specific binding with the ligands or the receptors, each of which has been bound to one of the porous adsorptive regions of the biochemical analysis unit, the receptor or the ligand being thereby specifically bound to at least one of the ligands, each of which has been bound to one of the porous adsorptive regions of the biochemical analysis unit, or at least one of the receptors, each of which has been bound to one of the porous adsorptive regions of the biochemical analysis unit, and
iii) detecting the receptor or the ligand, which has thus been specifically bound to at least one of the ligands or at least one of the receptors, by the utilization of a labeling substance,
wherein, at a stage between the step for subjecting the receptor or the ligand to the specific binding with the ligands or the receptors, each of which has been bound to one of the porous adsorptive regions of the biochemical analysis unit, and the step for detecting the receptor or the ligand, which has been specifically bound to at least one of the ligands or at least one of the receptors, by the utilization of the labeling substance,
at least one electrode is located such that the electrode is capable of causing an electric current to flow across each of the porous adsorptive regions of the biochemical analysis unit, and
a voltage is applied to the electrode in order to cause the electric current to flow across each of the porous adsorptive regions of the biochemical analysis unit.

2. An assay method using a biochemical analysis unit, comprising the steps of:
i) obtaining a biochemical analysis unit provided with a plurality of porous adsorptive regions, to which ligands or receptors have been bound respectively,
ii) subjecting at least one kind of an auxiliary substance-bound receptor or at least one kind of an auxiliary substance-bound ligand, to which an auxiliary substance has been bound, to specific binding with the ligands or the receptors, each of which has been bound to one of the porous adsorptive regions of the biochemical analysis unit, the auxiliary substance-bound receptor or the auxiliary substance-bound ligand being thereby specifically bound to at least one of the ligands, each of which has been bound to one of the porous adsorptive regions of the biochemical analysis unit, or at least one of the receptors, each of which has been bound to one of the porous adsorptive regions of the biochemical analysis unit,
iii) subjecting an auxiliary substance-combinable labeling substance, which is capable of undergoing specific binding with the auxiliary substance, to specific binding with the auxiliary substance of the auxiliary substance-bound receptor or the auxiliary substance-bound ligand having been specifically bound to at least one of the ligands, each of which has been bound to one of the porous adsorptive regions of the biochemical analysis unit, or at least one of the receptors, each of which has been bound to one of the porous adsorptive regions of the biochemical analysis unit, and
iv) detecting the auxiliary substance-bound receptor or the auxiliary substance-bound ligand, which has been specifically bound to at least one of the ligands or at least one of the receptors, by the utilization of the auxiliary substance-combinable labeling substance,
wherein, at a stage between the step for subjecting the auxiliary substance-bound receptor or the auxiliary substance-bound ligand to the specific binding with the ligands or the receptors, each of which has been bound to one of the porous adsorptive regions of the biochemical analysis unit, and the step for subjecting the auxiliary substance-combinable labeling substance to the specific binding with the auxiliary substance of the auxiliary substance-bound receptor or the auxiliary substance-bound ligand having been specifically bound to at least one of the ligands or at least one of the receptors, and/or
at a stage between the step for subjecting the auxiliary substance-combinable labeling substance to the specific binding with the auxiliary substance of the auxiliary substance-bound receptor or the auxiliary substance-bound ligand having been specifically bound to at least one of the ligands or at least one of the receptors, and the step for detecting the auxiliary substance-bound receptor or the auxiliary substance-bound ligand, which has been specifically bound to at least one of the ligands or at least one of the receptors, by the utilization of the auxiliary substance-combinable labeling substance,
at least one electrode is located such that the electrode is capable of causing an electric current to flow across each of the porous adsorptive regions of the biochemical analysis unit, and
a voltage is applied to the electrode in order to cause the electric current to flow across each of the porous adsorptive regions of the biochemical analysis unit.

3. A method as defined in claim 1 wherein the value of the voltage applied to the electrode is adjusted for each of the adsorptive regions.

4. A method as defined in claim 2 wherein the value of the voltage applied to the electrode is adjusted for each of the adsorptive regions.

5. A method as defined in claim 1 wherein the biochemical analysis unit is electrically connected to the ground.

6. A method as defined in claim 2 wherein the biochemical analysis unit is electrically connected to the ground.

7. A method as defined in claim 3 wherein the biochemical analysis unit is electrically connected to the ground.

8. A method as defined in claim 4 wherein the biochemical analysis unit is electrically connected to the ground.

9. A cleaning apparatus for cleaning a biochemical analysis unit provided with a plurality of porous adsorptive regions, to which ligands or receptors have been bound respectively, at least one kind of a receptor or at least one kind of a ligand having been specifically bound to at least one of the ligands, each of which has been bound to one of the porous adsorptive regions of the biochemical analysis unit, or at least one of the receptors, each of which has been bound to one of the porous adsorptive regions of the biochemical analysis unit, the apparatus comprising:
i) at least one electrode, which is located such that the electrode is capable of causing an electric current to flow across each of the porous adsorptive regions of the biochemical analysis unit, and
ii) control means for applying a voltage to the electrode in order to cause the electric current to flow across each of the porous adsorptive regions of the biochemical analysis unit.

10. An apparatus as defined in claim 9 wherein the control means is capable of adjusting the value of the voltage, which is applied to the electrode, for each of the adsorptive regions.

11. An apparatus as defined in claim 9 further comprising connection means for electrically connecting the biochemical analysis unit to the ground.

12. An apparatus as defined in claim 10 further comprising connection means for electrically connecting the biochemical analysis unit to the ground.
